(19) United States
(12) Patent Application Publication
(10) Pub. No.: US 2016/0024458 A1
(43) Pub. Date: Jan. 28, 2016

(54) Process For Inoculating Closed Photobioreactors With Cyanobacteria

(71) Applicant: ALGENOL BIOFUELS INC., Fort Myers, FL (US)

(72) Inventors: Laura BELICKA, Fort Myers, FL (US); Jessica BLANKS, Fort Myers, FL (US); Hayley FLORES, Fort Myers, FL (US); Isaac HOLOWELL, Fort Myers, FL (US); Frank JOCHEM, Fort Myers, FL (US); George MEICHEL, Fort Myers, FL (US); Harlan MILLER, III, Fort Myers, FL (US); Jesse Phillips-Kress, Fort Myers, FL (US); Brendan SCOTT, Fort Myers, FL (US); Christophe VASSEUR, Gruissan (FR)

(73) Assignee: ALGENOL BIOFUELS INC., Fort Myers, FL (US)

(21) Appl. No.: 14/774,459

(22) PCT Filed: Mar. 15, 2014
(86) PCT No.: PCT/US2014/029903

§ 371 Date: Sep. 10, 2015

Related U.S. Application Data
(63) Continuation-in-part of application No. 14/145,203, filed on Dec. 31, 2013, now Pat. No. 9,121,012.
(60) Provisional application No. 61/852,169, filed on Mar. 15, 2013.

(51) Int. Cl. C12N 1/12; C12P 7/06

(52) U.S. Cl. CPC C12N 1/12; C12P 7/065

(57) ABSTRACT

Methods of inoculum scale-up of cyanobacteria to commercial-scale closed photobioreactors for the production of a target molecule such as ethanol.

### CROSS-REFERENCE TO RELATED APPLICATIONS

This application claim priority to U.S. Nonprovisional patent application Ser. No. 14/145,203, filed Dec. 31, 2013. This application also claims the benefit of U.S. Provisional Patent Application Ser. No. 61/852,169, filed Mar. 15, 2013. The disclosures of these documents are incorporated herein by reference in their entirety.

### STATEMENT REGARDING FEDERALLY SPONSORED RESEARCH

This invention was made in part with United States government support under the Department of Energy grant number DE-EE0002867. The government has certain rights in this invention.

### REFERENCE TO SEQUENCE LISTING

Not Applicable.

### TECHNICAL FIELD

This invention relates to methods of scaling to commercial production closed bioreactors, including methods of rapid simultaneous inoculation of multiple commercial-scale closed photobioreactors. The process described herein is preferably directed to the use of cyanobacteria to produce target chemical products in photobioreactors.

### BACKGROUND OF INVENTION

Cyanobacteria form a phylogenetically coherent group of gram-negative prokaryotes that are capable of oxygenic photosynthesis, wherein their photosystems PSI and PSII extract and transfer electrons from water molecules to electron acceptors and generate oxygen as a co-product. They are capable of fixing carbon from CO₂ under aerobic conditions.

As photoautotrophic organisms, the rates of photosynthesis and growth of cyanobacteria are directly affected by the physical parameters of the environment.

In the wild, the competitive success of cyanobacteria depends on a continual fine-tuning of growth rate in order to exploit the changing nutritional environment. To cope with depleted nutrients and exploit those that are plentiful, the cyanobacteria undergo transitions from exponential to arithmetic (linear) growth into non-growth (stationary) physiological states.

The duration of the exponential and linear growth phase in culture depends upon the size of the inoculum, starting density of the inoculum, growth rate, environmental conditions, and capacity of the medium to support microbial growth. Cyanobacterial growth does depend on light intensity. The dependence on external light intensity is impacted by culture density.

It has been reported by Foster that wild cyanobacteria grow optimally in the range of 15-75 μM⁻² s⁻¹ and batch cultures progress from a lag phase into an exponential growth phase. This is typically followed by a period of linear growth that continues until the culture reaches the non-growing stationary phase. Linear growth in bacteria occurs when there are perturbations in the environment such that a critical nutrient is regulated arithmetically. In cyanobacteria, linear growth is most often associated with light limitation caused by self-shading of cells as cultures reach a certain cell density J. S. Foster, et al., Arch. Microbiol, (2007) 187:265-279. The optimal light range may be broader than indicated by Foster, such as 15-300 μE m⁻² s⁻¹.

In 1999, Deng and Coleman disclosed the introduction of new genes into the cyanobacterium Synechococcus PCC 7942 to create a novel pathway for fixed carbon utilization which created the target chemical product ethanol. M.-D. Deng and J. R. Coleman, Appl. Envir. Microbiology (1999) 65: 523-528. Related patents are R. P. Woods, et al. U.S. Pat. No. 6,306,639 and U.S. Pat. No. 6,699,696. Other target chemical products have been identified; see for example, U.S. Pat. No. 7,794,969 and U.S. Pat. No. 8,183,027.

In the production of target chemical products, such as ethanol from microorganisms such as cyanobacteria, an inoculum of the microorganism is needed so as to provide a population of such microorganism, suitable for scaling up to levels amenable to commercial scale production. In the case of specialty chemicals, produced in low amounts, this inoculum might be cultured in a vessel so that the culture density increases to a culture density suitable for reaching a production level that meets overall productivity metrics. [See for example PCT/US2011/022790, MICROORGANISM PRODUCTION OF HIGH-VALUE CHEMICAL PRODUCTS, AND RELATED COMPOSITIONS, METHODS AND SYSTEMS; see separately Example 1 of PCT/GB2012/050194] The production of other target molecules from cyanobacteria are discussed in Ruffing et. al., “Physiological effects of free fatty acid production in genetically engineered Synechococcus elongatus PCC 7942,” Biotechnology and Bioengineering (2012) 109:2190-2199; and in V. H. Work, et al., “Biocommodities from photosynthetic microorganisms,” Environmental Progress & Sustainable Energy (2013) 32:989-1001. In the case of commodity chemicals, such as biofuels, inoculum scale-up might proceed in several stages.

In the case of inocula to create cultures for open systems, published US application 20100304456 lays out some guidelines:

It is preferred that (1) the amount of biomass provided by the Closed Systems to inoculate the Open Systems should be equal to more than 5% of the carrying capacity of the aggregate Open Systems; (2) the growth rate of the species being cultivated is greater than approximately one and a half doublings per day (i.e. cell biomass doubles about every 16 hours); and that (3) no culture be maintained in any Open System for a period of more than 5 days. The combination of these three limitations assures that, under any circumstances, the culture should attain a biomass of the desired microbe that is equal to at least approximately 90% of the carrying capacity in 5 days or less. This is important for several reasons. First, a culture that is inoculated at a relatively high cell concentration (i.e. greater than 5% of carrying capacity) will dominate the medium compared to any unwanted cells that may have inadvertently been introduced. Second, because most species grow at rates substantially less than 1 doubling every 16 hours (1.5 doublings per day), a species that is capable of growing this rapidly will outpace most potential competitors. Third, the combination of the large inoculum (greater than 5% of carrying capacity) and high growth rate (greater than 1 doubling every 16 hours) assures that, within 5 days, the total biomass will be very near carrying capacity. These conditions are important to (1) reducing the risk of contamination, and (2) promoting the production of total biomass or the biosynthesis or production of oil. First, a potential contaminant would have to have a large inoculum and would have to grow more rapidly than the desired species to dominate the culture medium within 5 days. Second, oil production in particular is favored in cultures that are near carrying capacity because resources become limiting to growth once the culture passes 50% of carrying capacity. By limiting resources favorable to growth, one generally stimulates the biosynthesis of oil.

U.S. Patent Application Publication No. 2011/0217692 describes methods of controlling the temperature of a photobioreactor that is used to produce “carbon-based products of interest.” This process is designed to protect algae that are growing outdoors in the hot sunlight from overheating to the point of inhibition of cell growth or even cell death. In particular, a heat exchange chamber for containing a heat exchange fluid is disclosed, as is a thermal control layer between the reactor chamber and the heat exchange chamber. The photobioreactors typically have a culture thickness of from 5 mm to about 30 mm thick, and are typically placed flat either on top of the ground or on a water layer, with the heat exchanging chamber below. Various sterilization methods are described in paragraphs 81 through 91. In particular, paragraph 81 of U.S. Patent Application Publication No. 2011/0217692 shows the risks of contamination. In contrast, the present invention does not require the use of a temperature controlling layer and a heat exchange chamber.

Paragraph 82 of U.S. Patent Application Publication No. 2011/0287541 discusses amounts stored for use as inocula. However, in contrast to the present invention, the amount of inoculum to be used to inoculate a photobioreactor is not taught, the OD at which to transfer inoculum or at which to start the new culture is not taught, and the use of the carrying capacity of the system to determine the culture density at which to transfer cells or at which to inoculate cells, is also not taught by US2011/0287541.

Example 4 of PCT/AU2011/000829 describes inoculation of a large bioreactor with a volume of inoculum, followed by growth, and followed by further dilution.

Earlier art mentions the preparation of inocula ultimately for use in open, rather than closed, systems. For example, H. W. Blanche, Current Opinion in Biotechnology (2012) 23:390-395; E. Olguin, Biotechnology Advances (2012) 30:1031-1046; J. Quinn, Bioresource Technology (2012) 117: 164-171; I. Christenson, Biotechnology Advances (2011) 29:686-702 [discussing Cellana].

U.S. Pat. Nos. 7,770,322 and 8,268,601 both entitled “Continuous Batch Hybrid Process for Production of Oil and Other Useful Products from Photosynthetic Microbes,” disclose methods of continuous cultivation of photosynthetic cells as a first stage of culture growth. At a chosen OD, some culture is removed and replaced with fresh medium so that the growing culture has the same final volume (thus, a continuous culture) and remains in the same container. The culture that is removed is used to inoculate an outdoor open pond batch culture (thus, as stated in the title, it is a “hybrid” system, using both batch and continuous modes of culture growth). To avoid contamination, the open pond batch culture phase is only grown for a short period (5 days or less). In contrast, the present invention utilizes cyanobacteria that are grown in closed systems for several weeks or more. Also, the U.S. Pat. Nos. 7,770,322 and 8,268,601 patents teach that a high density inoculum should be used (“at least 5% of the carrying capacity” of the final system) so that the pond culture will be ready for harvest in about 5 days. In contrast, the methods of the present invention can utilize a smaller culture density of inoculum in the production photobioreactors, and this has significant impact on process economics at large commercial scales.

International Patent Application Publication No. WO 2012/101459 discloses processes for growing microalgae and cyanobacteria to produce metabolites. During the transition between the exponential growth phase and stationary growth phase, the cultures are stimulated to produce the metabolites such as lipids by treatment with a specific environmental stimulus: 1) a pH decrease below 6, followed by 2) a pH increase above 7, followed by 3) a high light intensity treatment. This publication discloses a starting culture density of algae inoculum (Synechococcus sp. and Synechocystis sp.) at 10⁶ cells per milliliter, taken from the sub-cultured algae which was transferred to a 50 L bioreactor. In contrast to WO 2012/101459, the methods of the present invention do not involve the use of a stimulus consisting of a pH decrease followed by a pH increase in order to switch the cells from a growth phase to a production phase.

International Patent Application Publication No. WO 2011/094457 discloses an inoculum of microorganisms comprising genetic modifications to make 3-hydroxypropionic acid or other compounds. Example 11a discloses that a bacterial culture was grown using a glucose feed in a stainless steel bacterial fermentor to an OD₆₀₀ of 3-4 at the first stage, and to an OD of about 5-6 at a second stage. During production of the compounds of interest, the inducer chemical IPTG was added when the cultures reached either an OD₆₀₀ of 2 (Example 11) or an OD₆₀₀ ranging between 15 and 20 (see Examples 11a, 11b, 11c). The cultures were then grown for about 48 hours prior to harvesting the compounds. The text of this publication relates to heterotrophic bacterial cultures grown in steel fermentors, rather than cyanobacteria grown in photobioreactors as is used in the present invention.

U.S. Pat. No. 7,794,969 discloses the production of n-alkanes in cyanobacteria using genetic modification by transformation. Example 3 mentions a 32 milliliter culture of OD(600)=0.1 which was grown to an OD=0.4. Further, Examples 5 and 8 disclose the transformation of host cells with foreign plasmid DNA in order to generate the genetically engineered strain. In this procedure, the transformation process occurred at an OD₇₃₀ of 1.0. However, this OD was only used for the specific situation of preparing the host culture for the DNA transformation procedure, not for scale-up of an inoculum culture to larger containers for production purposes. In contrast to the present invention, U.S. Pat. No. 7,794,969 does not disclose a suitable volume or culture density of inoculum to use in the scale-up preparation for production photobioreactor cultures.

Szita et al., 2005, “Development of a Multiplexed Microbioreactor System for High throughput Bioprocessing,” Lab on a Chip 5:819-826), discloses the use of pH sensors, DO sensors, and OD monitors to observe the growth of nonphotosynthetic microorganisms such as E. coli. Page 823, col. 1, lines 22-23 describes a starting inoculum density of OD₆₀₀=0.05. After 6 hours, the culture reaches stationary phase, and an OD₆₀₀=3.5 is obtained (page 823, col. 2, lines 48 through 51). Another nonphotosynthetic bioreactor type is noted to reach an OD₆₀₀ of 5.6 at stationary phase (page 824, col. 2, line 8). No photosynthetic organisms are discussed in Szita et al. Further, the OD that is measured is only at 600 nm. In contrast, the method of the present invention is performed with photosynthetic cyanobacteria in photobioreactors.

The present invention is directed to the creation of inocula suitable for introduction into closed bioreactor systems. The present invention provides a method for rapid scale-up of inoculum by monitoring of optical density, and control thereof. An embodiment of the invention permits the method to proceed by minimizing exposure of the inoculum to ambient air.

In another embodiment, a plurality of photobioreactors connected in parallel can be inoculated from a series of scale-up cultures. In a further embodiment, the culture is transferred rapidly and evenly to the plurality of photobioreactors. In a further embodiment, the process is performed so that the photobioreactor culture is axenic or substantially axenic.

### SUMMARY OF INVENTION

An aspect of the present invention is a method for producing cyanobacterial inoculum and transferring the inoculum to production photobioreactors to produce a product of interest. Another aspect of the present invention is a method for the staged growth of inoculum.

In an aspect of the invention, a process for inoculating a plurality of closed photobioreactors of determined carrying capacity for a genetically enhanced cyanobacterium is provided, by placing an inoculum of the genetically enhanced cyanobacteria in a first container through which light can pass, the container having a determined carrying capacity for the genetically enhanced cyanobacteria, to create an initial inoculum culture, growing the initial inoculum culture in the presence of light to an amount that can be from about 40% to about 60% of the carrying capacity of the first container to form a final inoculum culture, and transferring the final inoculum culture in the first container in a sterile manner to the plurality of closed photobioreactors, where the transfer occurs without exposure to air. The final inoculum culture in the closed photobioreactors can be, for example, at an amount less than about 5% of the carrying capacity of the closed photobioreactors. The first container can have, for example, an illuminated surface area to volume ratio of from about 1 to about 120 m²/m³. The first container can have, for example, an illuminated surface area to volume ratio of from about 70 to about 90 m²/m³. The final inoculum culture can be axenic. The OD₇₅₀ of the initial inoculum culture can be in the range of OD₇₅₀ of 0.01 to about 0.5. The closed photobioreactors can be, for example, vertical suspended photobioreactors made of polyethylene film. The genetically enhanced cyanobacteria can be capable of producing ethanol. The culture growing in the closed photobioreactors of can be grown, for example, from about 25 days to about 50 days prior to harvesting the product. The culture growing in the closed photobioreactors can be grown, for example, for more than 90 days prior to harvesting the product.

In another aspect of the invention, a process for inoculating a plurality of closed photobioreactors connected in parallel is provided, each photobioreactor having a volume from about 10 liters to about 100 liters, with a cyanobacteria genetically enhanced for the inducible formation of a target molecule, by growing an uninduced inoculum culture of the genetically enhanced cyanobacteria in a first container of pre-sterilized medium to a carrying capacity of from about 40 to 60%, and transferring the inoculum culture in the first container in a sterile manner to a plurality of production photobioreactors that contain pre-sterilized induction medium that allows the formation of a target molecule, where the transfer occurs at a pressure of from about 5 to about 50 psi without exposure to atmosphere, where the initial culture in the production photobioreactors can be at a density of less than about 5% of the carrying capacity of the final photobioreactors. The first container can have, for example, an illuminated surface area to volume ratio of from about 1 to about 120 m²/m³. The first container can have, for example, an illuminated surface area to volume ratio of from about 70 to about 90 m²/m³. The inoculum culture can be axenic. The OD₇₅₀ of the initial inoculum culture can be in the range of OD₇₅₀ of 0.01 to about 0.5. The closed photobioreactors can be, for example, vertical suspended photobioreactors made of polyethylene film. The genetically enhanced cyanobacteria can be capable of producing ethanol. The culture can be grown in the production photobioreactor, for example, for from about 25 days to about 50 days prior to harvesting the product. The culture can be grown in the production photobioreactor, for example, for more than 90 days prior to harvesting the product.

In an aspect of the invention, a process of making a product from a genetically enhanced cyanobacterial culture in a closed system is provided, by growing an inoculum of genetically enhanced cyanobacterial culture in a scale-up vertical photobioreactor from about 5 liters to about 50 liters with a culture width of from about ¾″ to about 10″ to a density of about 40% to about 60% of the carrying capacity of the scale-up vertical photobioreactor, transferring the genetically enhanced cyanobacterial culture to a production photobioreactor at an amount less than 5% of the carrying capacity of the final photobioreactor, and producing the product.

In yet another aspect of the invention, a process for inoculating a closed vertical bioreactor of less than 25 liters with a genetically enhanced cyanobacteria having an inducible promoter that controls the formation of a target molecule is provided, by placing the enhanced cyanobacteria, in an uninduced state, within an initial liquid culture of volume 0.1 liter to 1.0 liter into a first container of larger size, adding sterile liquid growth media into the first container by transferring the sterile liquid growth media under pressure without exposure to atmosphere in an amount larger than the volume of the initial liquid culture medium, and less than the container size to enable a growth to an OD of 1.5 to 2.0 to form a first inoculum liquid, and transferring the first inoculum liquid to the closed vertical bioreactor.

In another aspect of the invention, a process for inoculating a plurality of closed photobioreactors connected in parallel, each photobioreactor having a volume from about 10 liters to about 100 liters, with a cyanobacteria genetically enhanced for formation of a target molecule is provided, by growing an inoculum culture of the genetically enhanced cyanobacteria in a first container to an OD₇₅₀ of from about 1.0 to about 10.0 in a volume of from about 1 liter to about 10 liters of pre-sterilized medium, transferring the inoculum culture in a sterile manner under pressure without exposure to atmosphere from the first container to a pre-sterilized second container, growing the inoculum culture in the second container to an OD₇₅₀ from about 1.0 to about 10 in a volume of from about 20 liters to about 100 liters of pre-sterilized medium, transferring the inoculum culture in a sterile manner under pressure without exposure to atmosphere from the second container to a pre-sterilized third container, growing the inoculum culture in the third container to an OD₇₅₀ from about 2.0 to about 10 in a volume of from about 200 liters to about 1,000 liters of pre-sterilized medium, and transferring the inoculum culture in a sterile manner at a pressure of from about 5 to about 50 psi without exposure to atmosphere from the third container and a sterilized nutrient solution, to a plurality of pre-sterilized photobioreactors having a pre-sterilized liquid, where the pre-sterilized photobioreactors have inlet tubing and drip emitters configured in parallel from the third container, where the drip emitter controls the culture flow so that each of the photobioreactors connected in parallel can be inoculated at a substantially similar flow rate.

In an aspect of the invention, a process for inoculating a plurality of closed photobioreactors connected in parallel, each photobioreactor having a volume from about 10 liters to about 500 liters, with a cyanobacteria genetically enhanced for formation of a target molecule is provided, by determining conditions for growing the enhanced cyanobacteria in the exponential growth phase and in the linear growth phase in a first container, growing a first inoculum culture of the genetically enhanced cyanobacteria in the exponential growth phase or the linear growth phase in the first container to an OD₇₅₀ of about 1.0 to about 15, and such that the amount of genetically enhanced cyanobacteria in the first container can be in the range 40% to 60% of the carrying capacity of the first container, transferring the grown second inoculum culture in the absence of air to the plurality of closed photobioreactors, and forming the target molecule in the plurality of closed photobioreactors.

### DESCRIPTION OF THE FIGURES

FIG. 1. An embodiment of the inoculum transfer procedure.

FIG. 2. An embodiment of the inoculum transfer procedure.

The numbered figure elements for FIGS. 1 and 2 are:

100: Air Pump

101: Check Valve

102: Quick Disconnect Coupling

103: Tubing Cap

104: Culture/Media filled line

105: ⅜″ Santoprene Line to 80 L Inoculum bag

106: Luer Lock Air Pump Connection Fitting

107: 2 μm Filter

108: Ball Valve

109: Liquid Trap

110: Humidifier

111: CO₂ Delivery

112: Air Filled Line

113: CO₂ Delivery Line

114: 3 way valve

200: 1 L Vessel

300: 20 L Nutrient Carboy

400: Exhaust Trap

501; 502; 503 etc.: 5 L Vessel

FIG. 3. Schematic layout of inoculum embodiment. The numbered figure elements for FIG. 3 are:

1: Air inlet to pressurize vessel

2: 1 L culture with 500-900 ml culture volume

3: Large scale up vessel 1 (20 L, 80 L, or 500 L vessel)

4: Large scale up vessel 2 (80 L, 500 L, or inoculated PBR)

5: Media 1 (typically 20 L carboy with seawater and nutrients) to fill #3

6: Media 2 (typically 20 L carboy with seawater and nutrients) to fill #4

7: Overflow waste (if necessary)

8: Sample port

9: Peristaltic pump for better control on transfer without adding pressure to system

#3 (above) can be operated continuously to maintain an OD of 1.0 or lower. If culture reached full capacity, overflow can be turned over to new vessel (i.e., #4) or can be wasted in dump container (#7).

FIG. 4. Schematic layout showing scale-up to 4,500 liter bioreactor.

FIG. 5. Schematic layout of inoculum embodiment showing surge tank.

FIG. 6. Schematic layout of scale-up to large inoculum container, then transfer to a plurality of photobioreactors. The various sized containers, connecting tubing, pumping systems, and pressure compensation flow control devices (such as drip emitters) are shown.

The numbered figure elements for FIG. 6 are:

114: 3-way valve

601: Initial flask or vessel

603: First scale-up container

605: Second scale-up container

607: Third scale-up container

609: Plurality of photobioreactors connected in parallel

611: Pump

615: Pressure compensation flow control device (such as a drip emitter)

617: Gauge

FIG. 7 is a line graph showing cell growth (as measured by standardized OD₇₅₀) of several photobioreactor scale-up systems (9 liter flat panel systems, a vertical bubbled tubular system, and a 500 liter scale-up system) over a 48 day period. The 9 liter 1″ flat panel system has a much higher final OD than the 500 liter system.

FIG. 8 is a line graph showing the cell growth (as measured by OD₇₅₀ over a 31 day period) of ethanol-induced cultures growing in final production photobioreactors.

FIG. 9 is a table showing various parameters of several types of inoculum scale-up photobioreactors, including initial culture density in the scale-up cultures, the carrying capacity, the % of the carrying capacity at inoculum harvest, photobioreactor size, time (days) in scale-up or final system, and the starting culture density of the final production photobioreactor, calculated in terms of % of the carrying capacity of the final system.

### DETAILED DESCRIPTION OF THE INVENTION

The axenic (or substantially axenic) scale-up to large plots of photobioreactors (such as 1,000 or more) can have numerous logistical complications, such as possible contamination, sterilization of tubing and connection devices, sterilization of medium, multiple connections (each raising the risk of contamination of the system) along with issues related to the axenic transfer of the culture. Additionally, it is possible that large amounts of inoculum culture are forced to wait for an extended period of time in non-optimal conditions (such as high temperature, lack of mixing, poor gas exchange, poor light conditions) prior and during the transfer to the large-scale photobioreactors, which is likely to lower growth and productivity of the final photobioreactors. Further, the inoculum process can be time consuming and costly due to the additional personnel that would be needed for the process.

In an embodiment, methods of producing high quality inoculum are disclosed which can be used for commercial-scale production of compounds such as ethanol from cyanobacteria. Additionally, a process has been developed that enables large amounts of culture to be scaled-up and transferred to a plurality of photobioreactors. In an embodiment, the process results in multiple axenic or substantially axenic photobioreactors, each having a similar starting culture density. Further, the final transfer can occur rapidly. For example, 400 photobioreactors can be inoculated in about 30 minutes.

In another embodiment, methods of inoculating such that the % carrying capacity is between a specified range is utilized in order to optimize cell performance in the production reactor.

### DEFINITIONS

As used herein, the term “about” means approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) within a confidence interval of 90% or 95%.

As used herein, the term “organism” is used to refer to any species or type of microorganism, including but not limited to bacteria, yeasts and other fungi. As used herein, the term “antimicrobial” is used in reference to any compound which inhibits the growth of, or kills microorganisms.

The terms “axenic” and “pure culture” refer to a culture that contains cells that are all members of the same species or strain of organism.

The term “substantially axenic” refers to the condition in which one main organism is present, but the culture may be contaminated with a small amount (such as less than 0.01%, or 0.1%) of another organism. Preferably, the cyanobacterial culture is axenic. However, this is often difficult to achieve on a large scale basis, in an outdoor environment. Thus, in an embodiment, the cyanobacterial culture is substantially axenic—that is, a contaminant is present at very low levels, but there is no significant effect on cyanobacterial cell growth or product production.

The term “sterile” means free from living microorganisms. Thus, conventional sterile procedures or other effective procedures for sterilization may be used to insure that the culture medium, tubing and connection devices, containers, photobioreactors, pumps, and valves, are sterile or substantially sterile.

The term “substantially sterile” means substantially free from living microorganisms. For example, if a sterilization process removes all but about 0.001%, 0.01%, or 0.1% of the contaminants, it can be considered, in some situations, “substantially sterile”.

The term “pre-sterilized” means that the solution or object has been previously sterilized prior to use.

The term “in a sterile manner” as used herein refers to manipulations performed on sterilized or axenic liquids or objects so that they remain contaminant free. This can also be referred to as “sterile technique”. This may include, for example, techniques such as using a sterile laminar hood to transfer medium from one container to another, heat-treating or sterilant-treating edges of containers prior to transfer, and touching a culture (such as a plate) with only pre-sterilized tools. Preferably, an axenic culture that is transferred to another container “in a sterile manner” will remain axenic (contaminant free) when present in the new container.

The term “contaminant test medium” refers to any material which supports the growth or replication of at least one potential contaminating microorganism in the cyanobacterial culture. These types of media are often prepared from basal media such as nutrient broth or peptone water. The term “contaminant test medium” may also be used in reference to solid plated media, such as a contamination control broth-based solid medium which supports the growth of microorganisms. Also included within this definition are semi-solid and liquid microbial growth media.

The term “streaking” shall be understood to mean a method of inoculation of the surface of a solid medium such that, during subsequent incubation, individual bacterial colonies (rather than confluent growth) develop on (at least) part of the medium surface.

As used herein, the term “inoculum” refers to an axenic or substantially axenic culture of cyanobacteria that is to be used to start a new culture of lower density. The new culture may also have a larger volume, or the new culture may be a plurality of smaller volumes. An amount of inoculum is mixed with an amount of cell medium, and a new cell culture is grown.

The term “log phase growth” refers to the growth phase of cyanobacteria after the initial “lag phase” when the cells are multiplying exponentially by cell division. This can also be termed an “exponential growth phase”.

The term “linear phase” refers to the phase of growth after log phase but prior to stationary phase. Cell growth starts to slow down at this stage, but hasn't stopped. Nutrient limitation and light limitation are common causes of the initiation of this growth phase from the exponential phase of growth.

The term “stationary phase” refers to the phase of growth that occurs after both the log and linear phases where growth is attenuated. The onset of this phase can be caused by multiple factors, including depletion of nutrients, light limitation, or accumulation of products.

As used herein, the term “growth” is defined as expansion of the culture, i.e. increase of number of organisms in the culture, over a defined period of time.

As used herein, the term “OD₇₅₀” refers to the combination of the absorbance and the scattering of light having a wavelength of 750 nm. It is generally understood that while both absorbance and the scattering of light contribute to the OD, the scattering of light is the dominant factor for cyanobacterial cultures. The measurement is an estimate of culture density.

The term “biomass” as used herein refers to a mass of living or biological material and includes both natural and processed material.

The term “culturing” as used herein refers to incubating a cell or organism under conditions wherein the cell or organism can carry out biological processes.

The terms “culture medium” and “aqueous medium,” as used herein in reference to the growth of cyanobacteria, refer to a liquid having nutrients designed to support the growth of cyanobacterial cells. An aqueous medium can be derived from, for example, a natural water source such as a river, stream, lake, brackish water at the boundary between marine water and freshwater environment, or a marine water source. Aqueous medium, either freshwater or marine, can also be obtained from a well.

As used herein, the term “BG-11” means a standard cultivation medium for cyanobacteria that is well known to those of skill in the art (Rippka et al. (1979), “Generic assignments, strain histories and properties of pure cultures of cyanobacteria,” J. Gen. Microbiol. 111:1-61). BG-11 contains all of the nutrients required for growth of many species of cyanobacteria. BG-11 is sold by, for example, Sigma-Aldrich Co. LLC as the product “Cyanobacteria BG-11 Freshwater Solution” under SKU C3061. “MBG-11” (“Marine BG-11”) additionally contains NaCl, either from seawater, brackish water, or from salt addition to the medium to approximate the level that would be present in seawater. In an embodiment, NaCl is added to a level that is higher than that of seawater.

As used herein, the term “photobioreactor” means a device or system used to support a biologically active environment for the cultivation of photosynthetic microorganisms. A cyanobacterial photobioreactor is based on a photosynthetic reaction which is performed by the chlorophyll-containing cyanobacteria using dissolved carbon dioxide, water, and sunlight energy to form chemically reduced carbon-containing products from the carbon dioxide. The carbon dioxide is dispersed into the reactor fluid to make it accessible for the cyanobacteria. The photobioreactor comprises transparent or translucent material to permit sunlight to reach the cyanobacterial photosystems.

As used herein, the term “flexible film” means a continuous polymeric material or coating that is not structurally self-supporting, and preferably is at least partially translucent. Non-limiting examples of materials that can be used in flexible films suitable for use with the present invention are polyolefins, polyesters and vinyl copolymers thereof, including polyethylene, polypropylene, nylon and polyvinyl chloride.

The term “carrying capacity” refers to the maximum biomass or culture density a phototrophic/photosynthetic organism can achieve in a photobioreactor given the existing environmental conditions. This can be measured, for example, in terms of cell count (i.e., cells/ml), wet weight, dry weight, chlorophyll content, optical density at a chosen wavelength (such as, for example, OD₇₅₀), turbidity, or any other suitable means. Exemplary environmental parameters can include, for example, temperature, light intensity, mixing, gas exchange, nutrient level, length of light per day, thickness (width) and volume of the culture container, etc.

The term “percentage of the carrying capacity” of the system is a measurement of the percentage of the maximum culture density that can be achieved in the system given the existing environmental parameters. For example, if the carrying capacity is an OD₇₅₀ of 6, and the cells are removed at an OD of 3, then the cells are at 50% of the carrying capacity of the system. If the cells are removed at an OD of 1.5, then the cells are at 25% of the carrying capacity of the system. Similarly, cells that are inoculated to an OD of 0.6 would be inoculated at 10% of the carrying capacity of the system.

The photobioreactor may be made of rigid materials such as extruded plastic, molded plastic domes, or plastic sheets or panels, or flexible materials, such as plastic film, or a combination of flexible and rigid materials. It may include framing members to impart strength or form to materials such as plastic extrusion, panels, or film that would otherwise have inadequate mechanical properties to create the desired structure.

The photobioreactor may be fabricated from any material, including glass, but preferably a plastic that has the optical clarity to permit photosynthesis, can withstand long-term UV radiation exposure, as well as exposure to corrosive saltwater, heat, cold, expansion and contraction. Glass, and opaque or translucent plastics may also be used, as long they meet the needs of the photoautotrophic organisms to be grown in the system. Any person skilled in the art of thermoplastics can specifically design a plastic, or plastic mix which can be used for the photobioreactor tube. Virgin resins may be used to manufacture the tubes, but since cost is a likely significant factor, recycled plastics are preferred. A few of the particular plastics can include High Density Polyethylene (HDPE), PolyethyleneTerephthalate (PET), acrylic, Lucite, polypropylene and polycarbonate, as discussed in U.S. Pat. No. 8,586,353. The desired life-span of the photobioreactor is a factor in the decision of the type of material to use.

The photobioreactor is preferably transparent, translucent, or at least partially translucent. As used herein, “partially translucent” should be understood as permitting sufficient passage of light, particularly sunlight, into the photobioreactor to enable photosynthesis by photoautotrophic organisms within the photobioreactor.

As used herein, the term “translucent” means allowing light to pass through, with or without scattering of photons.

As used herein, the term “thermoplastic” means a continuous polymeric material or coating that is rigid and substantially structurally self-supporting, and preferably is at least partially translucent. Non-limiting examples of thermoplastics suitable for use with the present invention are polycarbonate and polymethyl methacrylate.

As used herein, the term “sparging” means a process whereby a gas is bubbled through a liquid.

As used herein, the term “harvesting the product” refers to collecting the desired product made by the cyanobacteria. Cyanobacteria can be enhanced to produce compounds such as ethanol, butanol, propanediols, and many other products. In one embodiment, the product is ethanol. The product can also be cell biomass or a lipid. Various harvest methods can be used, depending on the type of product. For example, the culture medium can be separated from the cells, and the product can then be isolated from the liquid. Alternatively, the entire culture can be harvested, treated, and the product can then be extracted.

### Methods of Inoculum Scale-Up to a Plurality of Photobioreactors: Stepwise Scale-Up Using Several Scale-Up Bioreactors of Increasing Size

Although contamination with other bacteria or other organisms may not be a problem for some types of algal or cyanobacterial cultures, the use of cyanobacteria for the production of ethanol, which can be quickly consumed by contaminants in the culture, generally requires a contamination free or at least a low contamination level culture in order to produce high amounts of product.

To alleviate these issues, a method has been developed to inoculate multiple outdoor commercial-scale photobioreactors with genetically enhanced cyanobacteria in a relatively quick, convenient, and preferably axenic manner. The cells are grown from an initial plate, to a flask, and then to successively larger inoculum containers, using axenic culture methods (autoclaving, use of a laminar flow hood, etc.) as described herein. In an embodiment, an axenic culture is sequentially transferred to grow in a container that has a size of from about 1 to 10 liters, then the culture is axenically transferred to a container having a size of about 20 to about 100 liters, then to a container having a size of about 100 to 1,000 liters. This 100 to 1,000 liter container is grown to an OD₇₅₀ from about 2.0 to about 10.0. This culture is then axenically distributed to multiple pre-sterilized transparent or translucent photobioreactors, where the compound of interest (such as ethanol) can then be synthesized and eventually collected.

In an embodiment, the plurality of photobioreactors can be inoculated substantially simultaneously. In an embodiment, the transfers occur in a sterile manner, or in a substantially sterile manner. This inoculation of the plurality of photobioreactors can occur within 30 minutes to one hour. In an embodiment, about 400 photobioreactors are inoculated in about 30 minutes.

The number of photobioreactors that receive the inoculum can be chosen as needed depending on the volume and culture density of inoculum, the initial culture density that is desired in the plurality of photobioreactors, pump and tubing requirements, sterilization requirements, etc. The inoculum container can be delivered, for example, to 1, 2, 10, 25, 50, 100, 400, 500, 1,000, 1,500, 2,000, 3,000, 4,000 or more photobioreactors. In an embodiment, the inoculum is delivered to a large land area of photobioreactors, such as 1 acre, 10 acres, 50 acres, 100 acres, 1,000 acres, or more.

For optimal culture growth it is preferable to start each stage of the scale-up process with a culture having a high enough culture density to prevent a significant “lag phase” due to a low culture density of the newly transferred cells.

It is also preferable that the transfer to each subsequent stage takes place before the culture reaches stationary phase. Otherwise, there may be a significant lag phase once the culture is placed in the new medium. Accordingly, in an embodiment, the OD₇₅₀ of the cells to be transferred is chosen so that it is at a high enough to allow a suitable OD₇₅₀ of the next dilution, but not high enough to be at stationary phase. Preferably, the cells to be transferred are in an exponential phase of growth. The cells can also be in an early linear phase, or a linear phase of growth. Each time the inoculum is transferred to a larger scale-up container and diluted with the appropriate amount of sterile medium, it will have a new lower culture density (and, accordingly, a lower OD₇₅₀).

The initial transfer to the first scale-up container can be performed by pouring the axenic culture into the container (filled with sterile medium) using sterile techniques inside of a laminar flow hood, if desired, rather than by using a pump.

While smaller-volume culture transfers early in the process can be done by carefully pouring from one container to another in a laminar flow hood or by use of sterile tubing set at a height-difference to allow gravimetric flow, the later scale up stages typically utilize a pumping system. The pump 611 can be, for example, a diaphragm pump or a peristaltic pump. Each of the pumps can be of the same type, or can be different types. Preferably, the chosen pumps are capable of being sterilized multiple times without damage.

Additionally, the method evenly distributes the inoculum, so that each of the multiple photobioreactors has a similar starting culture density. In an embodiment, the multiple photobioreactors are inoculated in a substantially simultaneous manner. In yet another embodiment, a plurality of photobioreactors, such as 200, 400, 1,000 or more, can be inoculated within 30 minutes to an hour.

A schematic diagram of a process of inoculation to a final plurality of photobioreactors is shown in FIG. 6. In an embodiment, the transfer from each scale-up container to the final plurality of photobioreactors occurs substantially as follows. Axenic culture is placed in the initial flask or vessel 601 containing sterile medium, and then grown up to a suitable culture density.

The inoculum grown in this initial vessel is then transferred to a first scale-up container 603 containing an appropriate amount of sterile medium and grown up to a suitable culture density. In an embodiment, the first scale-up container has a volume from about 1 liter to about 10 liters. In a further embodiment, the first scale-up container has a volume of about 5 liters.

The inoculum grown in the first scale-up container is then transferred to a second, larger sized scale-up container 605 containing an appropriate amount of sterile medium and grown to a suitable culture density. In an embodiment, the second scale-up container has a volume from about 20 liters to about 100 liters. In a further embodiment, the second scale-up container has a volume of about 80 liters.

To demonstrate how the dilution of the culture with the next larger stage container affects the OD₇₅₀, a 50 liter volume of the culture from the above-described second container having an OD₇₅₀ of about 10, once transferred to a 500 liter container with the appropriate amount of medium, would be diluted to an OD₇₅₀ of about 1. Similarly, a 20 liter culture from the above-described second container, grown up to a density of 2, then transferred to a 200 liter container with the appropriate amount of medium, would be diluted to an OD₇₅₀ of about 0.2.

This vessel is then transferred to a larger third scale-up container 607 containing sterile medium and grown to a suitable culture density. In an embodiment, the third scale-up container has a volume from about 200 liters to about 1,000 liters. In a further embodiment, the second scale-up container has a volume of about 500 liters.

When the culture in the third scale-up container 607 reaches a suitable culture density, it can be transferred to the plurality of photobioreactors 609 connected in parallel. Two three-way valves 114 allow for the tubing connections to be sterilized. A pressure of flow gauge 617 can be used to monitor the flow of inoculum to the photobioreactors. In an embodiment, each of the plurality of photobioreactors has a volume from about 18 to about 60 liters. In an embodiment, each of the plurality of photobioreactors has a starting optical density of about 0.01 to about 0.5 OD₇₅₀. In an embodiment, each of the plurality of photobioreactors is a vertical photobioreactor. In an embodiment, the number of photobioreactors that are inoculated is from about 100 to about 1,000. In a further embodiment, the number of photobioreactors that are inoculated is about 400.

In yet another embodiment, the distribution process to each of the photobioreactors (609) occurs substantially evenly, whether the photobioreactor is close to the inoculum or relatively far away from the inoculum container. Inoculated photobioreactors connected in parallel preferably have a variation in initial OD₇₅₀ of less than 5%.

Thus, in an embodiment of the invention, axenic cyanobacterial inoculum is sequentially scaled-up to a large inoculum container that is then evenly distributed to a plurality of photobioreactors, preferably in a simultaneous manner, so that each of the photobioreactors has a substantially similar starting culture density. This similarity of culture density is difficult to obtain when using serial inoculation to multiple photobioreactors.

### Methods of Inoculum Scale-Up to a Plurality of Photobioreactors: Simplified Scale-Up Using Fewer Scale-Up Vessels

While the above method generally utilizes a stepwise transfer of culture from a first container, to a second container, to a third container, then to the plurality of production photobioreactors, the method can also be performed somewhat more simply, with less transfers from one container to another.

Thus, in another embodiment, rather than use a multistep, sequential process of scale-up, fewer transfers are used, with the inoculum density higher at each harvest, and a lower culture density of the starting inoculum of each new scale-up container. For example, a culture can simply be transferred at each stage from a plate, to a flask, to a 9 liter thin panel scale-up reactor, to the production photobioreactors.

The culture density of the inoculated plurality of photobioreactors can be adjusted as desired. A low culture density inoculum (such as, for example, an OD₇₅₀ of 0.01, 0.05, 0.10, 0.2, 0.5, etc.) can be used for the final photobioreactors, particularly if a greater amount of photobioreactors are to be inoculated with a lesser amount of inoculant. If the photobioreactor batch is grown for a longer term, such as 30 days or more, the low inoculum density may not have a considerable affect on product production. Thus, a low culture density inoculum can, in some situations, be somewhat less expensive overall, since more photobioreactors can be inoculated per inoculum batch.

Inoculation of cyanobacterial cells to a production scale facility requires a large amount of clean, healthy inoculum cells, as well as a large amount of space in which to grow these cells. Thus, this part of the production process can be expensive. However, by increasing the OD of the inoculum that is transferred to the photobioreactors, less inoculum is needed per each photobioreactor, and the system can become more cost effective. Additionally, the initial OD of the final production photobioreactor can be decreased so that less inoculum is needed per photobioreactor. While this may affect productivity of some types of short term systems (such as 5 days to a week), depending on the strain and the product produced, it may not have an effect on the quantity of the final product when the culture is grown for a longer amount of time, such as, for example, about 25 days, 30 days, 35 days, 40 days, 50 days, 60 days, 75 days, 90 days, or longer.

Thus, altering the OD of the system so that a high inoculum OD is reached before transfer to the final photobioreactors, and the final photobioreactors are also inoculated at a relatively low OD, a large amount of photobioreactors can be inoculated with each inoculum container. For example, the starting culture density of the production photobioreactors can be at an OD₇₅₀ from about 0.005, 0.01, 0.02, 0.03, 0.04, to about 0.5. The inoculum culture for these production photobioreactors can be grown, for example, to an OD₇₅₀ of about 2, 3, 4, 6, 8, 10, 12, 14, 16, 18, 20, or more. Table 1, below, demonstrates this concept.

TABLE 1
Increased efficiency of the inoculation process by altering OD at transfer
and the initial OD of the final production photobioreactor

| | | | | | Number of |
|---|---|---|---|---|---|
| | | | | | final |
| | | | | Volume of | photobioreactors |
| | | | | scale-up | that can |
| | OD at | | Total liquid | inoculum | be inoculated |
| Scale-up | transfer | Initial OD of | volume of | added to | with one |
| reactor | to final | final | each final | each final | inoculum |
| volume | photobioreactor | photobioreactors | photobioreactor | photobioreactor | scale-up |
| (liters) | (liters) | (liters) | (liters) | (liters) | reactor |
| 100 | 5 | 0.01 | 50 | 0.1 | 1,000 |
| 100 | 5 | 0.1 | 50 | 1 | 100 |
| 100 | 5 | 0.5 | 50 | 5 | 20 |
| 100 | 5 | 1 | 50 | 10 | 10 |
| 100 | 10 | 0.01 | 50 | 0.05 | 2,000 |
| 100 | 10 | 0.1 | 50 | 0.5 | 200 |
| 100 | 10 | 0.5 | 50 | 2.5 | 40 |
| 100 | 10 | 1 | 50 | 5 | 20 |
| 100 | 20 | 0.01 | 50 | 0.025 | 4,000 |
| 100 | 20 | 0.1 | 50 | 0.25 | 400 |
| 100 | 20 | 0.5 | 50 | 1.25 | 80 |
| 100 | 20 | 1 | 50 | 2.5 | 40 |

A larger culture density inoculum can also be used, if desired. This may be preferred when a short lag phase is desired, or during shorter culture runs where product production will be higher if the inoculation density is higher. The choice of inoculation density can also be determined by the light intensity. Some strains may not tolerate high light intensity at low inoculation density, possible causing cell death or a longer lag phase before the growth phase.

Optical density (OD) is a measure of optical absorbance, typically using a spectrophotometer. Optical density (OD) is the absorbance of an optical element for a given wavelength λ per unit distance. If the OD is e.g. measured at a wavelength of 750 nm, for example, it may be referred to as OD₇₅₀. Measuring the optical density of a sample is an indirect method of determining the number of cells present. The amount of light of a specific wavelength that is absorbed by a culture is related to the number of cells. Thus, the OD measurement can be used as a simple and fast method of determining the cell count in a culture. The measurement of OD₇₅₀ of a culture correlates with cell growth, culture biomass, wet weight of a culture, and dry weight of a culture. The OD measurement can also correlate with the cell count per volume. However, it is often more feasible to simply measure OD₇₅₀ as an estimate of culture density than to utilize other methods.

The optical density (OD) at 750 nm can be used as a parameter for the culture density of cyanobacterial cultures. This measurement is based on both absorbance and on the light scattering of particles (the cells) in solution. As opposed to a measurement purely based on absorbance, the measurement of light scattering in a spectrophotometer is device-dependent—differences in the optics of different photometers result in different values. A commercially available turbidity standard can be used to standardize the OD₇₅₀ measurement of different spectrophotometers. One type of commercially available turbidity calculation standard is the Formazin Turbidity Standard (Sigma, TURB4000). Example 5 demonstrates how a turbidity standard can be used to standardize OD₇₅₀ over a number of spectrophotometer types.

The wavelength of OD that is measured can vary somewhat, but the OD at 750 nm is preferable, as it is likely to be responsive to differences in culture density, but not other factors such as the amount of chlorophyll or carotenoids in a given culture.

### Carrying Capacity

The carrying capacity of a given photobioreactor, which is the maximum culture density that can be obtained in a photobioreactor given the existing environmental parameters, can depend on many factors, such as the strain, the temperature, the length of time of the culture run, the light level and light cycle, the gas exchange, nutrient levels, and the dimensions of the photobioreactor itself. The inoculum culture can be harvested at one stage to be scaled-up to the next stage when the culture reaches a certain percentage of its carrying capacity. Typically, by the time the culture reaches its carrying capacity, cell growth has slowed substantially. Transferring at the maximum carrying capacity would be likely to result in a lag phase when the cells reach the new medium.

Thus, it is preferable to transfer the cells at less than 100% carrying capacity. The culture can be transferred to the next scale-up step when the cell growth is at about 30%, 40%, 45%, 50%, 55%, 60%, 70%, 80% of the system's carrying capacity. Preferably, the cells are transferred at a % carrying capacity of from about 25% to about 75%. More preferably, the cells are transferred when the culture reaches about 30% to about 60% carrying capacity. Even more preferably, the cells are transferred when the culture reaches about 40% carrying capacity.

Harvesting the scale-up culture at 40-60% of carrying capacity of the respective culture vessel offers two distinct advantages over growing scale-up cultures to higher densities: First, culture growth continues to slow down significantly after cultures surpass this range of carrying capacity, resulting in continually increasing loss of time for biomass production; producing the same amount of biomass occurs much faster at lower culture densities. Secondly, the average light intensity that individual algal cells experience decreases exponentially with culture density. Consequently, algal cells in dense cultures are acclimated to much lower average light intensities than cells in a less dense culture. The light stress caused by the transfer of cells from a more or less dense scale-up culture to a much lighter initial culture in the production bioreactors upon inoculation of the latter, is significantly lower in cells acclimated to higher rather than lower average light intensities. Therefore, harvesting the scale-up culture at densities well below carrying capacity of the scale-up vessel provides for algal cells significantly better acclimated to cope with the transfer to high average light intensities inherent in the inoculation of production bioreactors; particularly when the latter are inoculated to less than 5%, especially in the case of less than 1%, of their respective carrying capacity.

While harvesting scale-up cultures at even lower densities, such as below 30% of the carrying capacity, could provide for algal cells even better acclimated to high light intensities, doing so would require a substantial increase in the overall size/volume of the scale-up operation to provide the same biomass, or number of cells, required to inoculate the production bioreactors. This amounts to an increase in scale-up operations, which can increase scale-up costs.

The carrying capacity measurement can also be useful when determining the amount of inoculum that is to be transferred to the final production photobioreactors. If less inoculum can be used, this can decrease costs and space needed for preparation of the inoculum. However, it is best to avoid the situation of adding so little inoculum that the cells become bleached or otherwise compromised after a few days in the new production photobioreactor. This can result in a long lag phase or even total death of the new culture. Thus, it is desirable to inoculate with a high quality inoculum that has not reached stationary phase, but is at a relatively high culture density. In this situation, less volume for transfer is needed, and more production photobioreactors can be inoculated with a relatively small amount of inoculum.

Accordingly, in an embodiment, the final production photobioreactors are inoculated with an amount of inoculum that is equivalent to from about 0.01%, 0.02%, 0.05%, 0.1%, 0.25%, 0.5%, 0.75%, 1%, 1.25%, 2%, 3%, 4%, or 5% of the final carrying capacity of the system. In many cases, because the inoculum quality is high, the inoculum cells have not reached stationary phase, and the inoculum culture is axenic (or substantially axenic), a lower amount of cells can be transferred to the final phase without a substantial effect on the amount of final product that is produced.

### Light Availability and Photobioreactor Dimensions

Light is an important component of the carrying capacity of the system. Any desired lighting system that allows algal growth can be used to grow the cyanobacterial cultures. For example, the photobioreactors can be placed outdoors where they receive ambient light. The photobioreactors can be placed indoors with artificial light, either on an on/off cycle (such as 12 hours on, 12 hours off; or 16 hours on, 8 hours off). The photobioreactors can also utilize ambient sunlight plus additional artificial light, if desired.

The carrying capacity of the system can also be affected by the amount of light that each cell receives. While mixing and light cycles play a role, the amount of light per cell is often related to the basic dimensions of the photobioreactor. For example, cells growing in a large volume photobioreactor may have much less light available per cell than cells growing in a flask or test tube.

### Surface Area to Volume Ratio in the Scale-Up Photobioreactors

Cyanobacterial cells can grow faster, can obtain a higher culture density, and can have a higher carrying capacity in a system having a higher ratio of the illuminated surface area to the volume. In photobioreactor systems with high surface area to volume ratios, a greater proportion of the volume of the culture is exposed to light. Thus volumetric rates of photosynthesis, carbon production, and biomass accumulation are increased, and higher biomass densities (mass biomass/vessel volume) can be achieved. Assuming the reactor is deployed such that light is incident on all sides but the bottom on which the reactor sits, the following calculations apply:

Volume (V)=H×L×W

5-sided Illuminated surface area (SA)=2×(H×L)+2×(H×W)+(L×W) where most light exchange is across the H×L faces (i.e., 2×(H×L)>>2×(H×W)+(L×W).

If the photobioreactor receives light from only one broad H×L face, the surface area would simply be:

1-sided illuminated Surface area (SA)=H×L

As an example of this calculation, an inoculum culture is grown in a suspended vertical reactor that has a short width (W) (i.e., about 2.54 cm or 0.0254 m), with an illuminated face of height H=45.7 cm (0.457 m) by length L=76.2 cm (0.762 m). The reactor has a working volume of about 8.85 liters (0.00885 m³).

Following the above calculations, if only one side is exposed to light (area=0.348 m²), then the illuminated surface area to volume ratio is 39.3 m²/m³:

1-sided SAN=(0.457 m×0.762 m)/0.00885 m3=39.3 m²/m³.

If the photobioreactor is illuminated from all sides except the bottom, then the illuminated surface area to volume ratio is 83.6 m²/m³:

5-sided SA/V=(2×(0.457 m×0.762 m)+2×(0.457 m×0.0254 m)+(0.762 m×0.0254 m))/0.00885 m³=83.7 m²/m³.

### Maintaining Axenic or Substantially Axenic Cultures

Regarding the cyanobacterial culture, it is desirable that the scale-up culture is axenic—that is, free from other strains or other organisms, such as bacteria or fungi. In an embodiment, the culture is a pure culture of genetically modified cyanobacteria. An axenic culture can be scaled-up without contamination, the cell growth can be faster, and the product that is produced can accumulate at higher levels because it isn't consumed by the contaminant. Thus, in an embodiment, in order for the growing cyanobacterial cultures to be axenic or substantially axenic, particularly throughout multiple culture transfers, the components of the system (containers, medium, connectors, etc.) are treated so that they are substantially free from contaminants.

In an embodiment of the invention, all of the components of the system are sterilized, so that organisms other than the genetically enhanced cyanobacterial cells in the system are not present or are present at very low levels. The components of the scale-up system, such as flasks, nutrients, liquids, containers, connectors, pumps, etc. are sterilized in a suitable manner. The choice of sterilization method can depend on costs, residual residue that may be toxic to the cyanobacteria, heat or chemical stability of the materials, and effectiveness of the sterilization method. The components so sterilized can be “sterile”—that is, a completely free of live organisms. Alternatively, the sterilization process can be “substantially sterile”—that is, a small amount of live organisms may remain, but the bulk of the contamination has been removed. In some situations, such as sterilization-in-place of outdoor tubing with chemical sterilants, the complete removal of all organisms is difficult, but the remaining small amount of organisms does not pose a problem for the cyanobacterial culture.

Containers can be sterilized, for example, by autoclaving, by ozonation, by gamma irradiation, etc. Additionally, a wide range of suitable sterilizing solutions may be used to sterilize portions of the system. Basically, any solution suitable to sterilize or disinfect a surface can be used. In an embodiment, the solutions are biological oxidants. In an embodiment, the sterilizing solutions are halogen containing compounds, such as, for example, chlorine dioxide, bromine oxide, bromine chloride, monochloroamine, bromic acid, hypochlorous acid, chlorates, chlorites, hypochlorites, iodine monochloride, iodine trichloride iodine monobromide, etc. Combinations of two or more suitable compounds can also be used.

Further, the limited number of connections that are made, disconnected, and connected again during this process helps to mitigate the likelihood of introducing contaminants into the system.

The culture medium can be a freshwater medium or a saltwater medium, depending on the organism. In an embodiment, the medium is MBG-11 medium, but other medium types can be used. The liquid (freshwater, brackish water, or seawater) to be used for the culture can be sterilized along with or separately from the medium components.

Medium components are typically sterilized by autoclaving. Larger amounts of liquid can be sterilized by filtration, steam treatment, ozonation, or autoclaving. However, any suitable, effective means for sterilizing the medium in a way that doesn't cause toxicity to the cyanobacterial cells once they are added can be used.

The sterile medium for each of the steps can be added either before the inoculum is added, or it can be added to the container after the inoculum is added. Alternatively, the appropriate amount of water (either freshwater, saltwater, or brackish water) can be sterilized separately from the nutrient mix, and the two are added to the new culture container, either before, during, or after the addition of the axenic culture.

In some instances, it is more efficient to sterilize medium in a larger size container, and then transfer a small portion of it to the smaller container. When the smaller container is inoculated and allowed to grow to a desired OD, this volume can be transferred back to the remainder of the liquid in the larger container, to be allowed to grow to a desired OD, before being transferred to the next step. In this way, one less connection is made, thus there is a lower risk for contamination. For example, a larger amount of medium (such as 500 liters) can be sterilized in the third scale-up container that has previously been connected and sterilized together with the smaller container (such as 80 liters). In this example, 70 liters of sterile medium from the 500 liter container is transferred to the smaller container, 10 liters of inoculum is added, and the culture grows to a desired density. Then, the 80 liters of culture is inoculated into the 500 liter container by moving it back through the same sterile connection.

### Photobioreactor Types and Components

The connecting tubing that can be used is preferably thermoplastic tubing that can be autoclaved, is ozone resistant, and can withstand the wear of a peristaltic pump. In an embodiment, tubing material such as flexible PVC, Santoprene™ thermoplastic vulcanizate (TPV), C-flex®, can be used.

In an embodiment of the invention, the parallel closed photobioreactors are suspended vertical photobioreactors made of a transparent or translucent flexible film, and supported, for example, by a metal or wood support structure. These photobioreactors can be made to a suitable height, length, and width, as desired. A heat sealing apparatus can be used to form the edges of the photobioreactor.

In another embodiment, the parallel closed photobioreactors are horizontal bags made of transparent or translucent flexible film. These photobioreactors can be placed directly on the ground, on a platform, or can be floated on a water surface.

In an embodiment, a pressure compensation flow control device 615 is placed upstream of each of the plurality of photobioreactors 609 so that a similar amount of inoculum can be transferred to each photobioreactor. In an embodiment, drip emitters are used for this purpose. Exemplary drip emitters include, for example, pressure compensating drip emitters made of ozone-resistant material that have a pressure range between 5-60 PSI and a liquid flow rate between 0.5-and 5 GPM. Drip emitters of various sizes and materials can be obtained from irrigation supply stores. An example of a drip emitter is the Rain Bird XB-05PC ½ gph Xeri-Bug Emitter (Barb Inlet), or the DIG 06-011 FC 0-10 GPH Adjustable 8 Stream Drip Emitter ¼ in. barb, both of which are available on the world wide web at sprinklerwarehouse.com. Preferably, the drip emitters are made of a material that is sterilizable. In an embodiment, the drip emitters are re-used multiple times, with a sterilization procedure between each use.

The types of pumps that can be used to provide positive pressure without exposure to atmosphere include, for example, positive displacement pumps, such as peristaltic pumps, diaphragm pumps, piston pumps, and the like. Preferably, the chosen pump can withstand multiple sterilization passages, such as by steeping with a sterilant or ozonation.

Relative to US published application 20100304456, which teaches an inoculum equal to more than 5% of the carrying capacity of the final system, the present invention teaches as low as 1%, or lower. Further, with initial OD₇₅₀ of as low as 0.1, the present invention can achieve a doubling time of as low as 9 hours, and on average 12-14 hours.

### Cyanobacterial Strains

The systems described herein can be used to produce compounds of interest from cyanobacterial strains. Exemplary cyanobacterial genera that can be grown using the methods described herein include, but are not limited to, Synechocystis, Synechococcus, Acaryochloris, Anabaena, Thermosynechococcus, Chamaesiphon, Chroococcus, Cyanobacterium, Cyanobium, Dactylococcopsis, Gloeobacter, Gloeocapsa, Gloeothece, Microcystis, Prochlorococcus, Prochloron, Chroococcidiopsis, Cyanocystis, Dermocarpella, Myxosarcina, Pleurocapsa, Stanieria, Xenococcus, Arthrospira, Borzia, Crinalium, Geitlerinema, Halospirulina, Leptolyngbya, Limnothrix, Lyngbya, Microcoleus, Cyanodictyon, Aphanocapsa, Oscillatoria, Planktothrix, Prochlorothrix, Pseudanabaena, Spirulina, Starria, Symploca, Trichodesmium, Tychonema, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, Iyengariella, Stigonema, Rivularia, Scytonema, Tolypothrix, Cyanothece, Phormidium, Adrianema, and the like.

In an embodiment, the cyanobacterial strain is Cyanobacterium sp. “ABIcyano1” which is a proprietary strain of the genus Cyanobacterium. A deposit of the Algenol Biofuels Inc. proprietary strain of Cyanobacterium sp. ABICyano1, disclosed herein and recited in the appended claims has been made with the American Type Culture Collection (ATCC), 10801 University Boulevard, Manassas, Va. 20110. The date of deposit was Nov. 9, 2012. The ATCC Accession Number is #PTA-13311. The deposit includes 25 2-ml vials, each containing about 1.5 ml of cryopreserved cyanobacterial cells at a concentration of about 2.39×10⁷ cells per mL. All restrictions will be removed upon granting of a patent, and the deposit is intended to meet all of the requirements of 37 C.F.R. §§1.801-1.809. The deposit will be maintained in the depository for a period of thirty years, or five years after the last request, or for the enforceable life of the patent, whichever is longer, and will be replaced as necessary during that period.

This strain (Cyanobacterium sp. “ABIcyano1”) is tolerant of high light intensities and high temperatures. The strain also grows relatively quickly, and is relatively resistant to contamination by microorganisms. The strain tolerates a wide range of salinities. The strain contains an endogenous, 6.8 kb plasmid. Because of its hardiness, this strain may be a good choice of a cyanobacterial host organism for scale-up production of products such as ethanol.

In another embodiment, another cyanobacterial strain, Synechocystis sp. PCC 6803, is cultured using the methods described herein. The strain is publicly available through ATCC (strain designation number #27184).

In yet another embodiment, the cyanobacterial strain of Synechococcus sp PCC 7002 can be cultured using the methods described herein. The strain is publicly available through ATCC (strain designation number #27264).

Additionally, the cyanobacterial strain of (Synechococcus elongatus #PCC 7942 can be cultured using the methods described herein. The strain is publicly available through ATCC (strain designation number #33912).

### EXAMPLES

### Example 1

### Preparation of Contamination Test Medium and Solid Test Plates

The contaminant test medium was prepared as follows: To 500 mL filtered seawater, the following chemicals were added: 1 g peptone, 1 g yeast extract, 1 g glucose, 1 g sucrose, and 1 g amicase. The mixture was stirred until completely dissolved, then autoclaved at 121° C. for 30 minutes. The solution was then cooled to room temperature. Then, 20 mL of sterile 50×BG-11 and sodium thiosulfate pentahydrate stocks were added to the solution using a UV laminar flow hood.

Solid plates containing the contaminant test medium were prepared as follows: In a one liter bottle with a stir bar, 10 grams of Bacto Agar were added to 500 mL of RO water, and autoclaved at 121° C. for 30 minutes, then cooled slightly. The sterilized contaminant test medium liquid broth (above) was mixed in under sterile conditions inside a laminar flow hood, and plates were poured in a UV laminar flow hood. Plates were cooled and stored at 4° C. until ready for use.

### Example 2

### Quality Control of Contamination Test Medium Plates

For a positive control of contaminated test plates, an agar plate made from the contaminant test medium in the above example was placed on the laboratory bench top with the lid off for 10 minutes at room temperature. The lid was then placed back on the plate, the plate was wrapped with 3M micropore tape, and the plate was incubated at 30° C. for one week. In a typical test run, contaminant growth (presence of colonies) on this positive plate was observed within 2-3 days. In situations where no contaminants were observed with this positive control, the positive control failed, the plates were discarded, new plates were prepared, and the method was performed again until positive colonies were observable.

### Example 3

### Validation of Sterility Measures of Containers, Pumps, Tubing, Etc. Prior to Contact with the Cyanobacterial Cultures

Each step and component in the scale-up system was sterilized by some means. Smaller volumes of liquid and smaller sizes of tubing, connectors, and containers, were sterilized using an autoclave. When larger amounts of culture medium were used, the master nutrient mix was sterilized using an autoclave and added to well water that had been previously sterilized by other means (such as ozonation). The tubing, pumps, emitters, and connections were sterilized by either autoclave, ozonation, steeping in a sterilant, or by other suitable means. Each of these steps was initially validated to confirm that the method resulted in contaminant-free components. The steps were also checked intermittently throughout scale-up to confirm the lack of contaminants.

In order to validate the sterility of the various methods, a sample sterilized component was contacted with contaminant test medium broth for 2 days to 1 week at room temperature. Cloudy contaminant test medium broth indicated that the sterilization process was not complete. Clear contaminant test medium broth indicated that the sterilization process was successful.

### Example 4

### Testing for Contamination of a Cyanobacterial Culture Using the Contaminant Test Medium Plates

Contaminant test medium agar plates stored at 4° C. were warmed to room temperature. One drop (40 to 80 μl) of culture was streaked across a contaminant test medium agar plate using a sterile loop tool inside a laminar flow hood. The plate was then wrapped with 3M micropore tape and incubated at 30° C. for one week in the dark. Contamination, when present, was typically observed within 1-2 days. Inoculum cultures that were found to be positive for either bacterial or fungal contamination were discarded.

Outdoor photobioreactors were then tested at inoculation and intermittently during the production period. While some minorly contaminated cultures were able to make product with no observable decrease in output caused by the contamination, other cultures were more fully contaminated, and were then discarded.

### Example 5

### Standardization of OD₇₅₀ Measurement Using Different Spectrophotometers

As mentioned earlier, the OD₇₅₀ measurement can differ when different spectrophotometers are used, particularly since it is a measurement of light scattering rather than absorbance. To standardize the OD₇₅₀ measurements across several different spectrophotometers, a commercially available turbidity standard, Formazin Turbidity Standard (Sigma, TURB4000) was used. Several different dilutions of the standard were made, as shown in Table 2, below (n=10). The formazin standard was pre-diluted 1:2 with distilled deionized (“dd”) water prior to the dilutions shown in Table 2. The dilutions were measured in 5 different types of spectrophotometer.

TABLE 2
dilutions of the pre-diluted Formazin standard

| | Formazin standard | dd water (particle |
|---|---|---|
| Dilution factor | 2000 NTU | free) |
| 0.25 | 500 μl | 500 μl |
| 0.225 | 450 μl | 550 μl |
| 0.2 | 400 μl | 600 μl |
| 0.175 | 350 μl | 650 μl |
| 0.15 | 300 μl | 700 μl |
| 0.125 | 250 μl | 750 μl |
| 0.1 | 200 μl | 800 μl |
| 0.075 | 150 μl | 850 μl |
| 0.05 | 100 μl | 900 μl |
| 0.025 | 50 μl | 950 μl |

From the resulting measurements, conversion factors were determined for each type of spectrophotometer in order to standardize them to one selected spectrophotometer (Shimadzu UV-2450) to ensure the comparability of all optical density measurements, using the following calculation:

### Calculation of Conversion Factors:

Linear regression applied [b=0 (regression forced through point (0,0))]:

y1=m1×(UV-2450)

y2=m2×(test Spec)

To make the test spec value (y2) equal the UV-2450 value (y1)

y1=(m1/m2)*y2

Where (m1/m2)=the conversion factor

The slopes of the optical density values versus the dilution factors were calculated for the linear range, using the seven measurements from dilution 0.025 to 0.175. For the calculation, the regression was forced through zero. The slopes were then related to that of the UV-2450 results, and factors for conversion were calculated. The factor allows the conversion of OD₇₅₀ values measured with different spectrophotometers to the OD₇₅₀ value measured with the UV-2450 (Shimadzu) spectrophotometer, which was defined as the standard.

TABLE 3
Calculated conversion factors for OD₇₅₀ using various types of
spectrophotometers

| Spectrophotometer Type | Conversion factor |
|---|---|
| Shimadzu UV-2450 | 1.000 |
| Shimadzu UV-1800 | 1.236 |
| NovaSpec Plus 1 | 0.766 |
| NovaSpec Plus 2 | 0.901 |
| GENESYS 10S | 1.545 |
| Beckman Coulter Inc. DU 730 | 1.426 |

Note that the OD₇₅₀ measured in the examples herein have not been corrected with the standardization multiplier. The values listed in the examples are raw values from a Thermo Fisher Scientific Genesys 20 Visible Spectrophotometer, model 4001/4). Using methods shown in this example, one can standardize the OD₇₅₀ of any spectrophotometer to a given standard so as to have a consistent standardized OD₇₅₀ when using different spectrophotometers.

### Example 6

### Conversion of Several Measurements of Cell Growth

Although the OD is the fastest way to measure culture density or cell growth, other units of measurement, such as dry weight, wet weight, cells per ml, chlorophyll content, or other measurements, can also be used for measuring the culture density of a given system or the carrying capacity (i.e., the maximum culture density) of a given system. One of normal skill in the art can determine the conversion rate between the OD₇₅₀ and the other culture density measurements, such as cells/ml, for a given strain. Because the cells of different strains have different sizes, and different amounts of chlorophyll, the following conversions can be used to determine culture density at a given standard OD₇₅₀:

TABLE 4
Conversion between 1 unit standard OD₇₅₀ and Other Cell Growth
Measurements:

| | | | Chlorophyll | |
|---|---|---|---|---|
| | | | (mg/L | |
| | Standard | | extracted | |
| Strain | OD₇₅₀ | #cells/ml | chlorophyll) | Dry Weight |
| Cyanobacterium | 1.0 | 1.93 × 10⁸ | 4.4 | 270-337 mg/L |
| sp. ABIcyano1 | | | | dry |
| | | | | weight |
| Synechococcus | 1.0 | 1.4 × 10⁸ | 3.8 | 227 mg/L dry |
| PCC 7002 | | | | weight |
| Synechocystis | 1.0 | 4.3 × 10⁷ | — | — |
| PCC 6803 | | | | |

### Example 7

### Sequential Scale-Up of Inoculum

The initial inoculation of the inoculum scale-up system was performed in a sterile laminar flow hood by transferring 100 mL of culture into a 1 L bottle in the scale-up system, using a sterile pipet. This culture was then diluted with sterile MBG11 media until there is approximately 600-900 mL in the bottle. All further transfers were performed according to inoculum scale-up system protocols (i.e. pressurization of culture bottle or media bottle and opening of valves without breaking the sterile envelope).

### Transfer to 5 L Bottle

The 1 L bottle contained approximately 900 mL of culture. When this volume reached an ideal optical density at 750 nm (OD₇₅₀) of 3.0 (or higher, provided the culture is not in stationary phase), it was transferred to the previously connected three 5 L bottles. Each 5 L bottle received 300 mL of inoculum. The 5 L bottles were then topped with 2 L of MBG11, from the previously attached sterile medium carboy. This top-off resulted in an OD₇₅₀ of approximately 0.6. When this 2 L of culture reached an OD₇₅₀ of 2.0, it received another 2 L of medium (resulting in a final volume of 4 L at OD₇₅₀ of 1.0).

The medium may be varied to extend exponential and linear growth phase such that the optical density at 750 nanometers can exceed 4.0.

### Transfer to 80 L Flat Panel Reactor

When the culture in a single 5 L bottle reached an OD₇₅₀ of 3.0, it was transferred into an 80 L reactor by fusing the tubing on the bottles and the reactor with the sterile tube fuser. 20 L of medium were then added (also using the sterile tube fuser) for an OD₇₅₀ of 0.5. When this volume reached an OD₇₅₀ of 2.0, an additional 20 L of medium were added for an OD₇₅₀ of 1.1. Once again, when this culture reached an OD₇₅₀ of 2.0, it received the final 40 L of medium (OD₇₅₀ of 1.1).

### Transfer to 500 L Reactors

When the 80 L reactor reached an ideal OD₇₅₀ of 3.5, it was transferred into a 500 L reactor by means of the sterile tube fuser with 420 L MBG11 medium (resulting in an OD₇₅₀ of 0.6).

### Transfer to 4,500 L Reactors

When a 500 L reactor reached an OD₇₅₀ of 4.5, it was transferred to a 4,500 L commercial photobioreactor with the additional 4,000 L of MBG11 medium (final OD₇₅₀ of 0.5).

### Example 8

### Protecting Inoculum from Contamination

In previous work, the transfer of inoculum into 1 liter and 5 liter containers was done in a fume hood, which required extra time and space. The repeated need to use the fume hood represented a bottleneck in the process of growing up inoculum. Furthermore, more frequent manipulations increased the risk of contamination. One has to do work sterilely, and any time one opens up a flask there is always a risk of contamination.

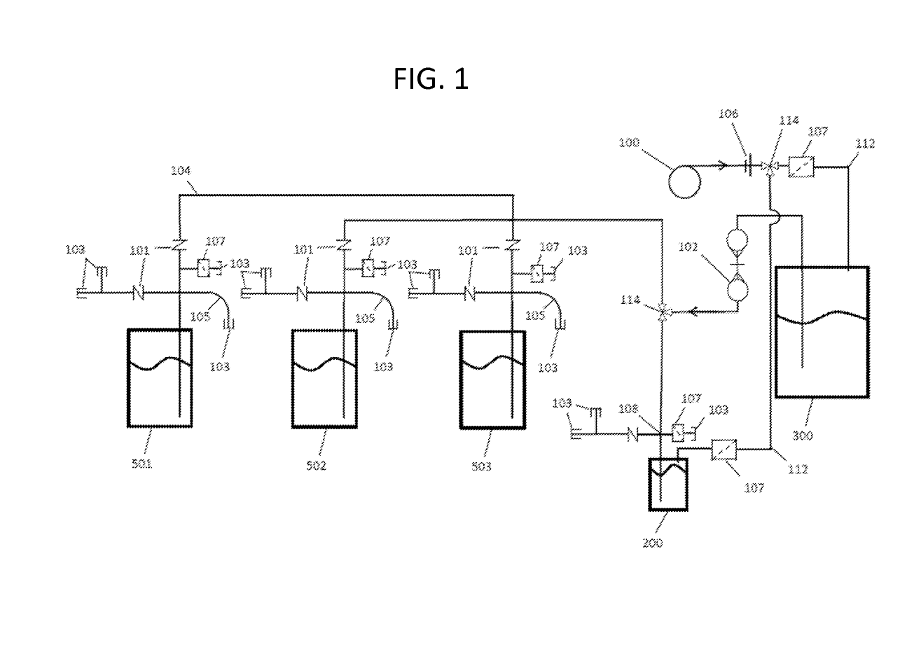

In one embodiment, depicted schematically as a system in FIG. 1, the one liter bottle and the five liter bottles were autoclaved as an interconnected system. The 20 liter carboy was autoclaved separately, but then connected aseptically to system in the hood or by sterile tube-fused connections outside of the hood. There was a transfer of the initial inoculum, in the hood or by sterile tube-fusing, to the one liter container. All connections were made and removed in the hood, (which, alternatively, can be mobile, or by remote sterile tube-fused connections). Ports were sprayed with ethanol and opened under flame from a portable burner (An alternative is to steam the ports). With the use of the sterile tube fuser, no further work was done in the hood after the initial 100 mL culture volume was added to the 1 L bottle.

Pressure from a pump was used to move liquid media. Excess gas volume left the system through an outlet port fitted with a 0.2 μm filter, such bacteria were unable to enter the system. There was a sterile input port, which allows input of compounds such as vitamin B-12 and neomycin. A sterile sampling port was present in the 1 liter and 5 liter vessels to remove liquid samples under pump pressure, so that one could determine the optical density of the inoculum culture. As the culture density increased within the 1 liter container, sterile sea water liquid was moved from the carboy to the 1 liter container to top off the liquid level in a way to maintain a desired optical density. As the liquid volume within the 1 liter container approached 1 liter (generally around 900 mL), the inoculum from the 1 liter container was moved via pump pressure to the five liter container.

The inoculum was allowed to grow in the 5 liter container, with addition of media via pump pressure done in a way to roughly maintain a desired optical density. This optical density was less than 1.0 for higher growth rates.

A sterile tube fuser allowed the transfer of inoculum culture from the 5 liter container to an 80 liter container. The liquid level was topped off to maintain a desired optical density. There was also a sterile tube fuser which allowed transfer of the inoculum culture from the 80 liter container to a 500 liter container.

In further prophetic embodiments, a plurality of one liter containers can be operationally connected to a plurality of 5 liter containers operationally connected to a plurality of 80 liter containers operationally connected to a plurality of 500 liter containers.

### Example 9

### Staged Inoculum Up to 4,500 Liter

Using the procedure outlined in Example 8, 900 mL of liquid were transferred to a 1 liter bottle. The 900 mL comprised 150 mL of culture and 750 mL of medium and nutrients.

After obtaining an OD of 2.0, the 900 mL of culture were transferred via pumping according to Example 8 into two 5 liter bottles with 2 liters of medium in each. When the OD within the 5 liter bottles reached 1.5, another liter of medium was added to each bottle. When the OD once again reached 1.5, an additional 2 liters of medium were added to each 5 liter bottle, for a total volume of 4 liters in each bottle.

After obtaining an OD of 3.0, the 8 liters of culture were transferred via pumping according to Example 8 into 20 liters of natural saltwater within an 80 liter bioreactor supported on a frame. Each time the OD reached 2.0, the volume was doubled until a volume of 80 liters was reached.

After obtaining an OD of 2.5 in the volume of 80 liters, the culture was transferred via pumping according to Example 8 into a bioreactor of volume 200 liters, which had sequential media additions until a final volume of 500 L was reached.

After obtaining an OD of 5.0 in a volume of 500 L, the culture was transferred into a bioreactor of volume 4,500 liters.

The 1 liter, 5 liter, and 80 liter containers used 1% CO₂ as a carbon source for growth. The larger containers utilized 1.75% CO₂. The 1 liter, 5 liter, and 80 liter containers were cultivated in a greenhouse. The added liquid comprised BG-11 (with 304 EDTA) and Na₂CO₃ (20 mg/Liter)+K₂HPO₄ (40 mg/Liter).

### Example 10

### Another Example of Inoculum Stain Up to 4,500 Liter

Using the staging of inoculum in a manual procedure and similar to the outline in Example 7, 600 mL of liquid were transferred to a 1 liter bottle. The 600 mL comprised 100 mL of culture and 500 mL of medium and nutrients.

After obtaining an OD of 2.5 or up to 4.0, the 600 mL of culture was transferred via pumping according to Example 7 into 10 liters of natural saltwater within an 80 liter bioreactor supported on a frame. The 10 L culture volume was subsequently topped-off to 80 liters with sterile saltwater and nutrients over the course of 5 days, while maintaining the OD at levels between 0.5 to 1.0.

After obtaining an OD of 1.0 in a volume of 80 liters of culture, the 80 L volume of inoculum was transferred via pumping according to Example 7 into a bioreactor having a volume capacity of 500 liters at an OD containing 100 liters of sterile seawater and medium. The total volume was 180 liters with a final OD ranging between 0.2 to 0.5. After obtaining an OD of 1.0 in the initial volume, the initial volume was subsequently topped-off to 500 liters of sterile saltwater and nutrients over the course of 7-10 days, while maintaining the OD at levels between 0.5 and 1.0.

After obtaining an OD of 4.5 in a volume of 500 liters of culture, 500 L of culture is transferred into a 4,500 liter bioreactor.

The 1 liter and 80 liter containers used 1.2% CO₂ as a carbon source for growth. The larger containers utilized 1.75% CO₂. The amount of CO₂ is dependent on the restriction of the diffuser and the height of the water column. The 1 liter and 80 liter containers were exposed to light from Spectralux T5 fluorescent 54 W (about 100 PAR at initiation 0.1 to 0.25 OD; and 400 PAR at OD 2.5 or greater). The added liquid comprised BG-11 (with 304 EDTA) and Na₂CO₃ (20 mg/Liter)+K₂HPO₄ (40 mg/Liter) 15 mg/L NaNO₃.

### Example 11

### Relating to Inoculation of Multiple Bioreactors: Treatment with Sterilizing Gas

Groups of bioreactors are preferably designed so that a sterilizing gas (such as ozone) can be supplied to all components of the system including each bioreactor and associated piping.

The piping systems are preferably free of dead ends and each system should end in a 0.2 μm filter so that the sterilizing gas (e.g., ozone) can be drawn across the entire internal surface of each system. Points where sterilizing gas will be added to the system should be identified and filters sized appropriately to ensure a consistent flow of 25 LPM with less than a 5 PSI pressure drop at these inlet points.

As a general matter, contamination can be controlled with the use of ozone. As to liquid water, this can be done by injecting concentrated ozone gas into process seawater. As to bioreactors and process lines in need of control, this can be done by treatment with humid ozone gas. Initially obtained sterility can be maintained by keeping the system isolated by 0.2 μm air filters during liquid transfers.

As to sterilization of seawater by ozone, the TRO [total residual oxidant] content of seawater will degrade from about 10 to about 0 in about 1 to 4 days, depending upon factors including liquid volume, light intensity, and temperature. The TRO of treated seawater should be sufficiently low so as not to interfere with the inoculation process, and preferably about zero. As a practical matter, ozone treated seawater should be allowed to stand so that TRO can decay for no less than 48 hours before inoculation.

Further as to the use of ozone, pressure is preferably maintained between 5 and 30 psi in the ozone generator to maintain its functionality. Flowpaths in the seawater sterilization system are aligned and checked to ensure that pumps do not work against closed valves.

### Example 12

### Relating to Inoculation of Multiple Bioreactors: Use of a Manifold

A manifold can be used to connect multiple photobioreactors (PBRs) in series (i.e., header) for inoculating each PBR from a single source culture and making a single connection to each reactor.

Separately, as a prophetic example, a manifold may be used in reverse for connecting multiple source cultures; however, a homogeneous stream is not made and tubing diameters must be considered. The term manifold can reference a series of connections for linking more than one vessel to a single line that can be used for splitting a large volume of liquid (or gas) into multiple smaller vessels or conversely, combining multiple small source volumes into a larger reservoir. By connecting multiple vessels to a single line, there is only one connection made to the PBR at time of inoculation.

### Example 13

### Relating to Inoculation of Multiple Bioreactors: Use of a Mixing Chamber

Inoculation of research and commercial photobioreactors requires a large volume or biomass of potentially axenic cyanobacteria culture. In order to meet the volume or biomass demands, multiple cultures were often mixed together into a single chamber or linked together in series.

Processing a single chamber to meet the capacity of inoculum for sterility and cleanup is not always practical or feasible for the specific inoculation event. Linking cultures in series also is not ideal for creating a homogenous culture, largely due to inconsistencies in mixing ratios between the linked cultures. This prophetic embodiment describes a practice for and verification of using a mixing chamber for up to three source cultures to create a homogenous stream for inoculation of replicate photobioreactors. More than three source cultures may be used as long as the combined tubing inner diameters (ID) of the mixing chamber (and source culture harvest line) exceed the mixing chamber outlet tubing ID.

The mixing chamber is positioned as an intermediate vessel between multiple source cultures that flow into the chamber through three separate inlets and an outlet vessel for depositing the mixed stream via a single outlet siphon. The outlet is connected to a peristaltic pump that draws head through the sealed mixing chamber, which creates a siphon from the source cultures at equal rates. The inlets extend further than the outlet siphon into the mixing chamber to allow for adequate mixing below the outlet siphon level. The multiple source cultures enter the mixing chamber via the inlet lines and become homogenized below the outlet siphon level. Once the chamber volume reaches the outlet siphon, the chamber contents are homogenously mixed and the outlet stream is an average of all source culture cell densities. For example, if three source cultures are being used with the following cell densities measured as OD₇₅₀: 1.0, 1.5, and 1.75, the average culture density of the mixed source cultures should be 1.42 OD₇₅₀. Using a 10 L carboy has yielded sufficient results in mixing three 80 L source cultures at varied cell densities into a single homogenous stream.

The outlet line can be any diameter and more inlets also can also be installed on the mixing chamber; however, the outlet diameter cannot exceed the maximum of a single inlet or the sum of tubing ID's of all inlets combined. The inlet lines are connected to the lid by barbed fittings forming a union through the lid to extend into the chamber from the underside of the lid. The inlet extension into the chamber is preferably longer than the outlet line that extends through the chamber. The inlet lines are preferably the same length, although slight variations (<1 cm) in the lengths have not shown to affect results significantly. The outlet line is connected to the barbed fitting on the lid with the largest diameter, and also to the transfer tubing used in the peristaltic pump. The pump draws from the single outlet line coming out of the mixing chamber. The pump drawing force on the siphon tube with a larger diameter than the combined source culture harvest lines (i.e., inlet tubes) provides enough force to siphon from multiple source cultures. The source culture harvest line and mixing chamber inlet tube IDs should match; however these may be different as long as the maximum siphon tubing ID does not exceed the combined inlet tubing ID's and the chamber inlet IDs are not greater than the source culture harvest line IDs.

### Example 14

### Relating to Inoculation of Multiple Bioreactors: Use of a Surge Tank

In order to connect inoculum and nutrients to multiple bioreactors, one may employ an inoculation/nutrient delivery header along with a surge tank. Such a system can comprise a diaphragm pump and a sequence of valves and piping that can direct liquid flow to and from a surge tank (recirculation) through a variety of paths. This recirculation allows for equal liquid distribution to the reactors.

In one example, the system consisted of a fiberglass deck box and a polyethylene surge tank. The deck box contained the pump, piping and valves that allow for the directional control of liquid flow. The plumbing consisted of ½ inch schedule 40 PVC, ½ inch PVC ball valves, ½ inch PVC unions, ½ inch schedule 80 threaded tees, ½ inch PVDF and polyethylene barbed fittings.

The surge tank was located external to the deck box and acted as the re-circulatory reservoir for liquid additions. All santoprene lines on the surge tank were equipped with 25 mm 0.22 μM PTFE Polyfilters; each having a terminal male luer fitting, a three inch section of ¼ inch silicone tube with a slide clamp, and a male luer fitting secured to the filters female luer end. The silicone tubing can be attached to santoprene via a barbed polypropylene reducer. All barbed fittings were cable tied in place. This filter and clamp arrangement allowed air to be forced through the lines to clear any residual liquid without compromising the sterility of the system.

Additionally, one may transfer sterile seawater to the surge tank using a peristaltic pump. One may use a diaphragm pump to transfer inoculum culture during the inoculum scale-up. On may use a diaphragm pump to transfer organisms into the surge tank.

### Example 15

### Scale-Up of a Strain of Cyanobacterium sp. ABICyano1 to a Plurality of Outdoor Photobioreactors

A scale-up system for the simultaneous inoculation of 800 photobioreactors was developed and demonstrated. This system consisted of three stages of growth in liquid medium before being distributed to the final outdoor photobioreactor. The initial inoculum was a 100 mL of culture of the strain Cyanobacterium sp. ABICyano1 (genetically enhanced to produce ethanol when induced) in a 250 mL flask, grown on a shaker table, using a 16 hours on/8 hours off light cycle with a light intensity of 100 μE m⁻² s⁻¹.

This was transferred to the “first stage container”, which was a 5 L glass media bottle containing 4 L of culture, which was outfitted with santoprene tubing (⅜″ ID ⅛″ wall-thickness) for the purpose of using a sterile tube fuser to make a sterile connection for transfer to the next stage of growth. The light cycle for this stage was 16 hours on/8 hours off, with a light intensity of 100 μE m⁻² s⁻¹. The “second stage container” was a translucent, flexible plastic 80 L bioreactor that was housed in a metal and plastic frame and also includes santoprene tubing for transfer to the third stage. The “third stage container” was a flexible plastic 500 L reactor that was also housed in a metal and plastic frame and has a santoprene tubing line for culture input. The culture outlet of the 500 L reactor differed from the previous stage in that it consisted of 1″ braided PVC tubing that was used to make the connection to the inoculum delivery system at the block of 800 reactors, where there was also a section of 1″ braided PVC tubing.

At each stage, the OD was measured. Thermo Fisher Scientific Genesys 20 Visible Spectrophotometer, model #4001/4, using 1 cm cuvettes and set at an absorbance of 750 nm. The machine was automatically calibrated daily, and was also manually calibrated before each use with seawater that was used for the medium preparation. Culture samples were analyzed in an undiluted form until they reached an OD₇₅₀ of 0.5. Above that level, the samples were diluted as needed to keep the OD₇₅₀ below 0.5.

In this specific example of an inoculation of 800 photobioreactors from two 500 L reactors of genetically enhanced Cyanobacterium sp. ABICyano1 culture, there were two separate batches (batch A and batch B, below) that were grown up identically from the 250 mL flask stage to the 500 L reactor stage:

Batch A: Axenic Cyanobacterium sp. ABICyano1 culture was grown to an OD₇₅₀ of approximately 1.0 in a 250 mL flask and axenically transferred to an autoclaved 5 L media bottle using a laminar flow hood. The culture reached an OD₇₅₀ of 1.024 with a final volume of 4 L after 5 days of growth and was transferred to a gamma-irradiated 80 L vertical reactor. This transfer was completed using a sterile tube fuser to connect the santoprene tubing (⅜″ ID, ⅛″ wall-thickness) between the 5 L bottle and 80 L reactor and a peristaltic pump to transfer the culture from the 5 L bottle to the 80 L reactor. The culture reached OD₇₅₀ of 1.06 with a final volume of 44 L after 3 days of growth and was transferred to a gamma-irradiated 500 L vertical reactor using the same sterile tube fusing technique as the previous transfer. After 26 days of growth, the culture reached an OD₇₅₀ of 2.22.

Batch B: The same procedure was followed as for Batch A, above, except that the culture in the 5 L bottle reached and OD₇₅₀ of 0.97 before transfer, and reached an OD₇₅₀ of 2.096 in the 44 L container.

Batch A and Batch B, each with a final volume of 524 L, were transferred to a block of 800 vertical photobioreactors, each having a final volume of 18 liters (that is, 400 photobioreactors were inoculated from each batch).

Each 524 L reactor was then axenically connected to the inoculum delivery system on the 800 block by connecting the 1″ braided tubing from the reactor to the inoculum delivery system and steeping the surfaces of tubing that were exposed to atmosphere with a liquid sterilant. The inoculum delivery system and the delivery pathway to the 800 reactors were previously sterilized by ozonation. After the tubing was sufficiently sterilized, culture was delivered to the 800 reactors simultaneously using a diaphragm pump and piping system consisting of PVC and HDPE piping.

The upstream tubing of each of the 800 photobioreactors was fitted with a drip emitter, as shown in FIG. 6 (Woodpecker Pressure Compensating Junior with Barb; Manufacturer's No. 21502-001400; Model #: 01WPCJL4; Length—1.378″; Depth—0.037″; Width—0.052″; flow of 1 gallon/hour when pressure is between 7-45 PSI). Delivery pressure was maintained between 15-20 PSI in order to facilitate flow through the emitters, which have a pressure requirement of 7 PSI and a flow rate of 4 GPH. The parameters for this scale-up and inoculum delivery to the multiple photobioreactors is summarized in Table 5, below.

TABLE 5
Bioreactor scale-up parameters for Example 15

| | 250 mL | 5 L | 80 L | 500 L | 800 Block |
|---|---|---|---|---|---|
| Bioreactor Info | Plastic flask | Glass | Flexible Plastic | Flexible | 18 L hanging |
| | | media | Reactor | Plastic Reactor | vPBR |
| | | bottle | supported by | supported by | |
| | | | metal/plastic | metal/plastic | |
| | | | frame | frame | |
| Type of | n/a | Santoprene | Santoprene ⅜″ | Braided PVC | Braided PVC |
| Transfer | | ⅜″ ID | ID ⅛″ wall- | 1″ ID | 1″ ID up to |
| Tubing to next | | ⅛″ wall- | thickness | | Diaphragm |
| stage | | thickness | | | pump, PVC |
| | | | | | and HDPE |
| | | | | | piping to |
| | | | | | vPBRs |
| Method used to | Biohood | Peristaltic | Peristaltic pump | Diaphragm | n/a |
| Transfer to next | | pump | | pump | |
| stage | | | | | |
| Method of | Gamma- | Autoclave | Gamma- | Gamma- | Ozonation |
| Sterilization | Irradiation | | Irradiation | Irradiation | |
| Light Cycle | 16/8 | 16/8 | Ambient light | Ambient light | Ambient light |
| Light Intensity | 100 μE/m²/s | 150 μE/m²/s | Ambient light | Ambient light | Ambient light |

| | Flask | 5 L | 80 L | 500 L |
|---|---|---|---|---|
| Batch 1 | | | | |
| OD @ Transfer | ~1 | 1.024 | 1.06 | 2.224 |
| Volume @ | 100 mL | 4 L | 44 L | 524 L |
| Transfer | | | | |
| # of Days | n/a | 7 | 5 | 26 |
| Grown Before | | | | |
| Transfer | | | | |
| Batch 2 | | | | |
| OD @ Transfer | ~1 | 0.976 | 0.97 | 2.096 |
| Volume @ | 100 mL | 4 L | 44 L | 524 L |
| Transfer | | | | |
| # of Days | n/a | 7 | 5 | 26 |
| Grown Before | | | | |
| Transfer | | | | |

After the delivery to the 800 photobioreactors was complete (approximately 30 minutes), the OD₇₅₀ values for the 10 reactors sampled were very similar with a minimal standard deviation of 0.01, as shown in Table 6, below.

TABLE 6
The drip emitter delivery system results in similar inoculation densities
of the production photobioreactors

| | Starting |
|---|---|
| Photobioreactor # | inoculum OD₇₅₀ |
| 1 | .191 |
| 2 | .169 |
| 3 | .176 |
| 4 | .17 |
| 5 | .164 |
| 6 | .196 |
| 7 | .206 |
| 8 | .198 |
| 9 | .192 |
| 10 | .196 |

### Example 16

### Growth of Scale-Up Inoculum Culture to a High Density in Flexible Film Containers with a 1″ Culture Width

In order to achieve a fast growing, high density inoculum, the scale-up procedure was performed in vertical flexible polyethylene containers (“flat panels” or “bags”) having a relatively narrow culture width (about 1″) in order to optimize the amount of light per cell. Previous scale-up runs demonstrated that larger batches of inoculum cultures often resulted in lower final cell densities. This was due, in part, to the lack of light availability that occurred with a larger width in the larger containers, particularly at higher cell densities. The cells eventually became self-shading, causing slower cell growth.

In this scale-up system, the inoculum photobioreactors had the following dimensions: length: 30 inches; height: 18 inches; thickness (width): 1 inch. The total culture volume was 9 liters per bag. The flat panel system was composed of an 8 mil polyethylene bag held in place in a 1 inch wide steel frame, additionally using clear UV-stabilized polycarbonate side sheets to provide support for the polyethylene bag. The polyethylene bag was equipped with four ports, carrying barb connectors for tubing seamed into the side seams of the bag: a port for CO₂/air mix supply provided to the culture through a nitrile aquarium-type air diffuser rod mounted at the bottom of the culture bag; a port for venting exhaust air in balance with air/CO₂ supply volume; a port for adding sterile starter culture inoculum; and a port for harvesting the scale-up culture.

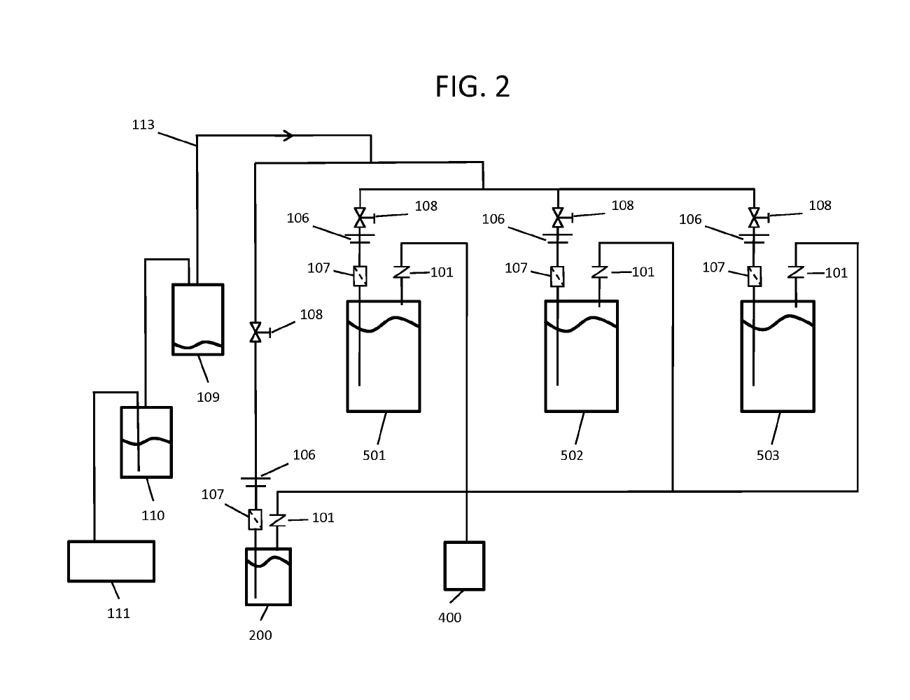

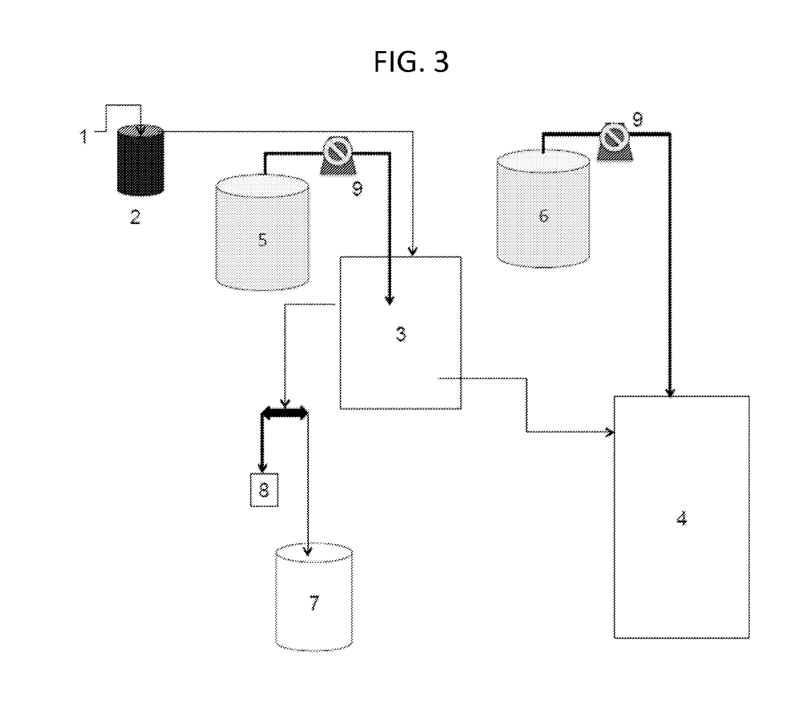

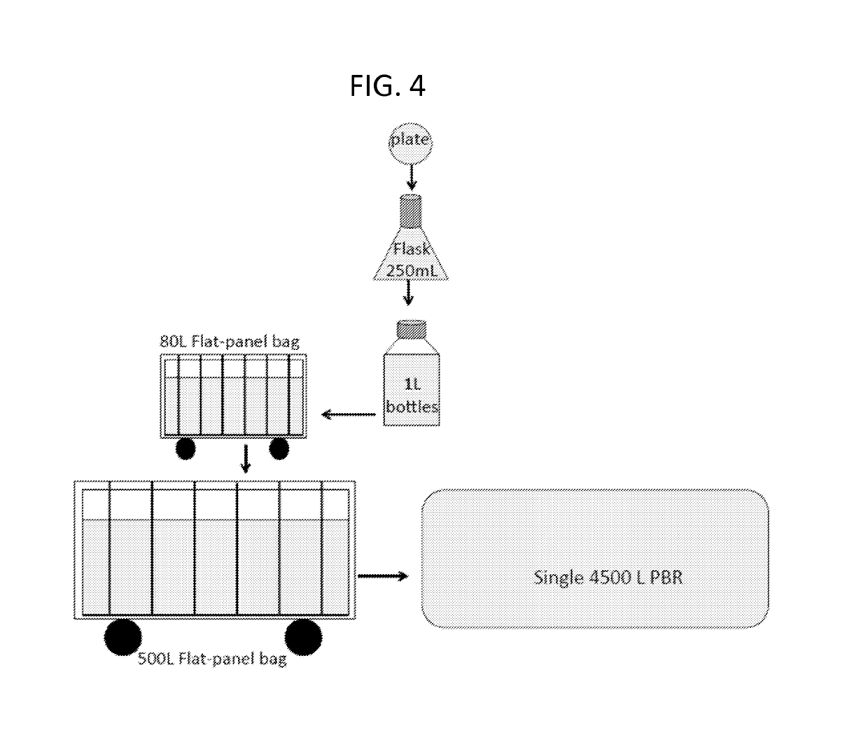

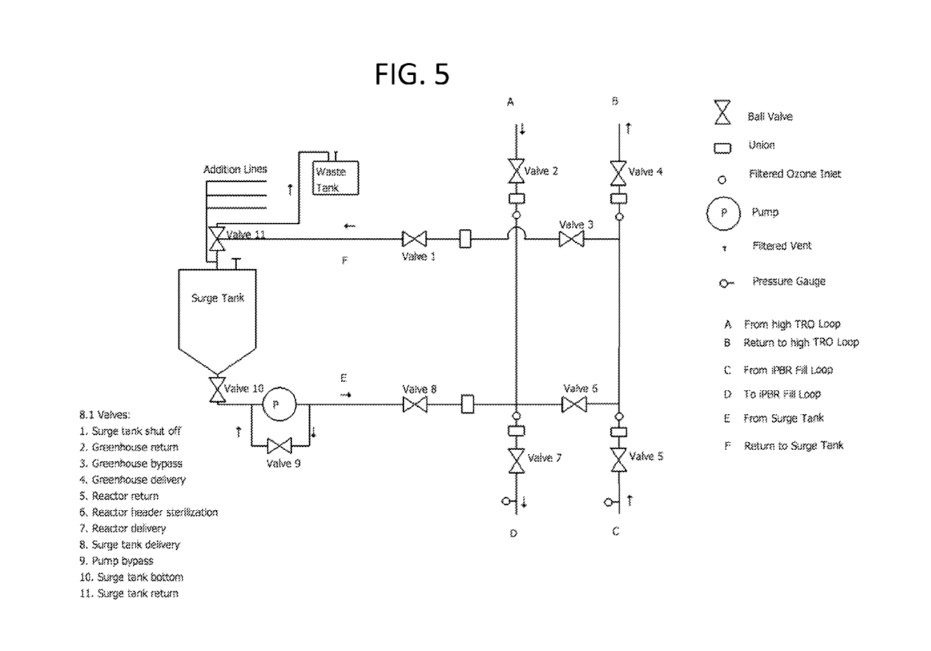

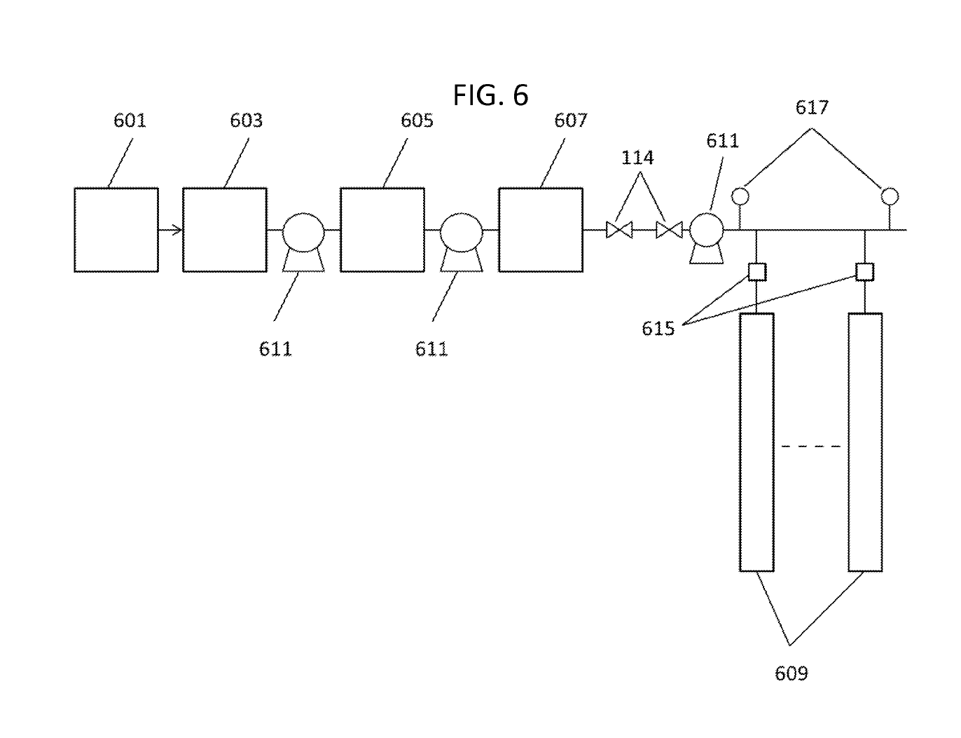

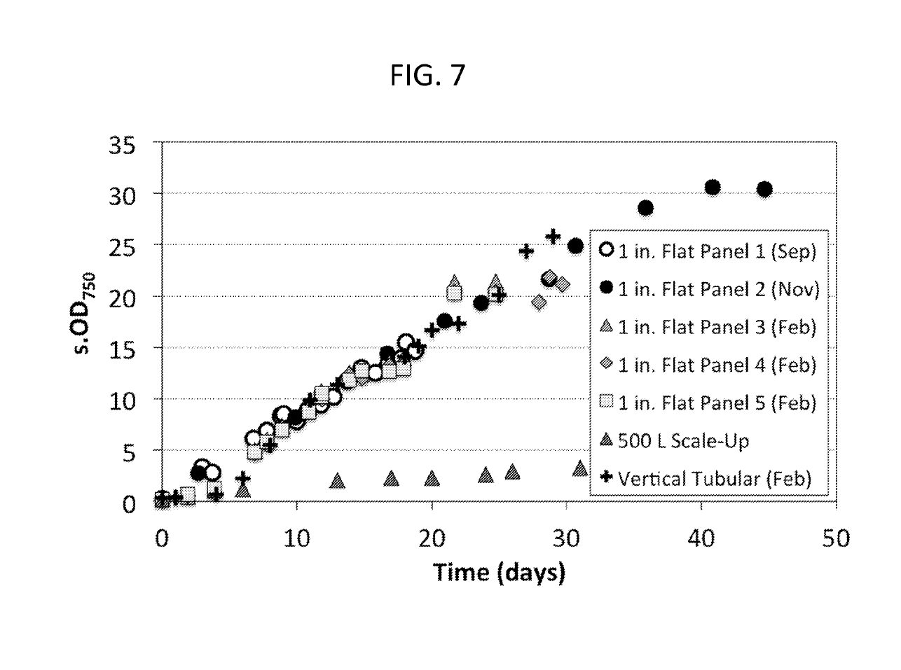

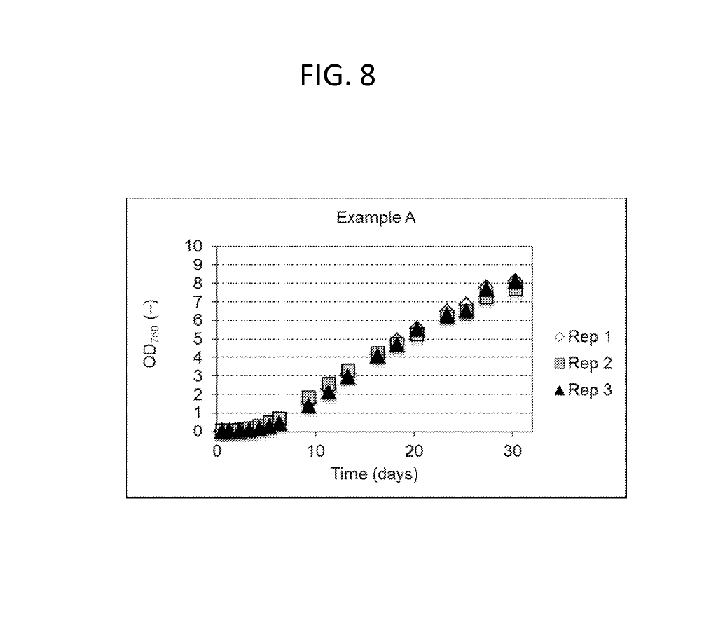

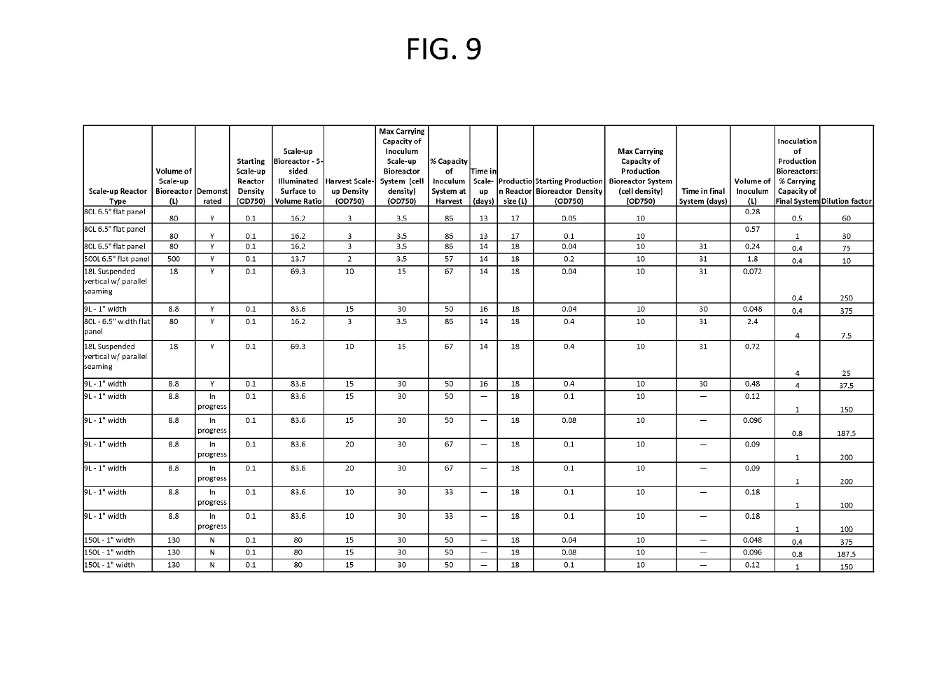

The polyethylene bags were suspended from metal frames with polycarbonate side sheaths to provide lateral support of the bag and to maintain a 1 inch culture width. The containers were fitted with inlet and outlet ports, as shown in FIG. 2. The polyethylene bags were sterilized by gamma radiation. These were then filled with autoclaved seawater (from an underground well) and amended with BG-11 nutrients except that the nitrate was replaced with 5.5 mM urea. No antibiotics were added.

The scale-up culture was started with a dense 5 L starting culture having an OD₇₅₀ of 3.0. The scale-up culture was run as a single batch. For every 5.5 OD₇₅₀ increase in culture density, another dose of BG-11 nutrients (with nitrate replaced with 3.0 mM of urea) was added to the culture. Cultures were maintained outdoors at ambient light and temperature and were bubbled with a constant mix of 2% CO₂ in air initially, which was increased to 4% CO₂ in air when the culture reached a density of 1 OD₇₅₀. The pH was maintained between 7.0 and 8.5. The total gas flow was 2 SLPM.

Two types of inoculum scale-up reactors were tested using the above method, with the following parameters and results:

As a first example, six separate 9 liter inoculum culture containers were grown with a relatively low initial OD₇₅₀ of 0.2. The carrying capacity of the system was 30 OD₇₅₀. This inoculum was grown to an OD of 15. Thus, the % of the total carrying capacity of the inoculum system at harvest was 50%. FIG. 7 is a graph showing the OD₇₅₀ of the scale-up photobioreactors over time.

This was then inoculated with an initial OD₇₅₀ of 0.04 to twelve 18 liter final production photobioreactors. The carrying capacity of final production system was 10 OD. Thus, the % of the carrying capacity of the inoculum was 0.04/10×100=0.4%. The ethanol concentration at day 32: 0.23% v/v.

As another example, a 9 liter inoculum culture container with a higher starting inoculation density of OD₇₅₀=2.2 was grown to an OD of 15. The carrying capacity of the inoculation system was 30 OD₇₅₀. Thus, the % of the total carrying capacity of inoculum system at harvest was 50%. FIG. 7 is a graph showing the OD₇₅₀ of the scale-up photobioreactors over time.

The culture was then inoculated to six 18 liter final production bioreactors at an initial OD₇₅₀ of 0.04. As above, this low inoculum density is about 0.04/10×100=0.4% of the carrying capacity of final production system (10 OD₇₅₀). The ethanol concentration at day 30 was 0.42% v/v. FIG. 8 is a graph showing the OD₇₅₀ of a final production photobioreactor over time.

### Example 17

### Growth of Scale-Up Inoculum Culture to a High Density in 1.5″ Wide Bubbled Columns

As another example of a suitable scale-up bioreactor system for producing high quality, high density inoculum, a multi-tubular vertical, air bubble-mixed growth system was used. The system comprised 20 separate 1.5 inch diameter vertical tubes assembled from clear PVC. The tubes were linked at the top with a header tube and at the bottom with a footer tube. The system was sterilized with ozone, and flushed with autoclaved seawater (from an underground well). The system was then filled with autoclaved seawater/well water amended with BG-11 nutrients (with the nitrate replaced with 5.5 mM urea). No antibiotics were used. The total liquid volume of the system was 53 liters. The system was inoculated from a 5 L start culture to an initial OD₇₅₀ of 0.35. The culture was run as a single batch. Every 7 days, another dose of BG-11 nutrients without nitrate and 5.5 mM urea was added to the culture. The culture was bubbled at 2 SLPM with air and 10% CO₂ addition under pH control at a setpoint of 7.3.

### Example 18

### Examples of Various Inoculum Density Schemes

The table in FIG. 9 shows various Scale-up photobioreactor schemes, with different volumes, inoculum density, carrying capacity, and time the culture spends in a given photobioreactor. Several of the exemplary systems have already been tested; some are in progress, and others have not been tested yet. The graph also shows how different dimensions can result in different illuminated surface area to volume ratios.

The graph measures the carrying capacity of the inoculum system as well as the approximate % of the carrying capacity at which the cells are harvested and sent to the next (final) stage of cultivation. The graph also shows the carrying capacity of the final production photobioreactors, and the % carrying capacity at which the final photobioreactors are inoculated.

### Example 19

### Surface Area to Volume Ratio in the Scale-Up Photobioreactors

A 45.7 cm×76.2 cm×2.54 cm inoculum scale-up photobioreactor was prepared and filled with 8.85 liters of liquid consisting of sterile medium plus an axenic culture of Cyanobacterium sp. ABICyano1. This strain was genetically enhanced to produce ethanol upon induction of an inducible promoter. During the scale-up phase, however, the ethanol production was repressed. Both sides of the photobioreactor were exposed to light. For this system, the illuminated surface area to volume ratio was calculated to be 83.7 m²/m³.

1. A process for inoculating a plurality of closed photobioreactors of determined carrying capacity for a genetically enhanced cyanobacterium, comprising the steps of:
a. placing an inoculum of the genetically enhanced cyanobacteria in a first container through which light can pass, said container having a determined carrying capacity for the genetically enhanced cyanobacteria, to create an initial inoculum culture;
b. growing the initial inoculum culture in the presence of light to an amount that is from about 40% to about 60% of the carrying capacity of the first container to form a final inoculum culture; and
c. transferring the final inoculum culture in said first container in a sterile manner to the plurality of closed photobioreactors, wherein said transfer occurs without exposure to air.

2. The process of claim 1, wherein the final inoculum culture in the closed photobioreactors is at an amount less than about 5% of the carrying capacity of the closed photobioreactors.

3. The process of claim 1, wherein the first container has an illuminated surface area to volume ratio of from about 1 to about 120 m²/m³.

4. The process of claim 1, wherein the first container has an illuminated surface area to volume ratio of from about 70 to about 90 m²/m³.

5. The process of claim 1, wherein the final inoculum culture is axenic.

6. The process of claim 1, wherein the OD₇₅₀ of the initial inoculum culture is in the range of OD₇₅₀ of 0.01 to about 0.5.

7. The process of claim 1, wherein the closed photobioreactors are vertical suspended photobioreactors comprised of polyethylene film.

8. The process of claim 1, wherein the genetically enhanced cyanobacteria are capable of producing ethanol.

9. The process of claim 1, wherein the culture growing in the closed photobioreactors of step c is grown from about 25 days to about 50 days prior to harvesting the product.

10. The process of claim 9, wherein the culture growing in the closed photobioreactors of step c is grown for more than 90 days prior to harvesting the product.

11. A process for inoculating a plurality of closed photobioreactors connected in parallel, each photobioreactor having a volume from about 10 liters to about 100 liters, with a cyanobacteria genetically enhanced for the inducible formation of a target molecule, comprising the steps of:
a. growing an uninduced inoculum culture of the genetically enhanced cyanobacteria in a first container of pre-sterilized medium to a carrying capacity of from about 40 to 60%;
b. transferring the inoculum culture in said first container in a sterile manner to a plurality of production photobioreactors comprising pre-sterilized induction medium that allows the formation of a target molecule, wherein said transfer occurs at a pressure of from about 5 to about 50 psi without exposure to atmosphere, wherein the initial culture in the production photobioreactors is at a density of less than about 5% of the carrying capacity of the final photobioreactors.

12. The process of claim 11, wherein the first container has an illuminated surface area to volume ratio of from about 1 to about 120 m²/m³.

13. The process of claim 11, wherein the first container has an illuminated surface area to volume ratio of from about 70 to about 90 m²/m³.

14. The process of claim 11, wherein the inoculum culture is axenic.

15. The process of claim 11, wherein the OD₇₅₀ of the initial inoculum culture is in the range of OD₇₅₀ of 0.01 to about 0.5.

16. The process of claim 11, wherein the closed photobioreactors are vertical suspended photobioreactors comprised of polyethylene film.

17. The process of claim 11, wherein the genetically enhanced cyanobacteria are capable of producing ethanol.

18. The process of claim 11, wherein the culture is grown in the production photobioreactor for from about 25 days to about 50 days prior to harvesting the product.

19. The process of claim 11, wherein the culture is grown in the production photobioreactor for more than 90 days prior to harvesting the product.

20. A process of making a product from a genetically enhanced cyanobacterial culture in a closed system, comprising:
a. growing an inoculum of genetically enhanced cyanobacterial culture in a scale-up vertical photobioreactor from about 5 liters to about 50 liters with a culture width of from about ¾″ to about 10″ to a density of about 40% to about 60% of the carrying capacity of the scale-up vertical photobioreactor;
b. transferring said genetically enhanced cyanobacterial culture to a production photobioreactor at an amount that is less than 5% of the carrying capacity of the final photobioreactor; and
c. producing said product.

21. (canceled)

22. (canceled)

23. (canceled)
